(19) United States
(12) Patent Application Publication
(10) Pub. No.: US 2025/0235533 A1
(43) Pub. Date: Jul. 24, 2025

(54) INJECTABLE PROLONGED-RELEASE HYDROGEL FORMULATIONS OF TOLL-LIKE RECEPTOR AGONISTS FOR INTRATUMORAL INJECTION TO INCREASE IMMUNOTHERAPY RESPONSE

(71) Applicants: Massachusetts Institute of Technology, Cambridge, MA (US); The Brigham and Women's Hospital, Inc., Boston, MA (US); The General Hospital Corporation, Boston, MA (US)

(72) Inventors: Robert S. Langer, Newton, MA (US); Carlo Giovanni Traverso, Newton, MA (US); Arijit Basu, Quincy, MA (US); Avik Som, Boston, MA (US); Jan-Georg Rosenboom, Cambridge, MA (US); Gabrielle Ndakwah, Cambridge, MA (US); Eric Wehrenberg-Klee, Boston, MA (US); Umar Mahmood, Winchester, MA (US)

(73) Assignees: Massachusetts Institute of Technology, Cambridge, MA (US); The Brigham and Women's Hospital, Inc., Boston, MA (US); The General Hospital Corporation, Boston, MA (US)

(21) Appl. No.: 18/417,024

(22) Filed: Jan. 19, 2024

(51) Int. Cl. A61K 39/39; A61B 18/00; A61B 18/02; A61K 9/00; A61K 39/00; A61P 35/00

(52) U.S. Cl. CPC A61K 39/39; A61K 9/0024; A61P 35/00; A61B 2018/00577; A61B 18/02; A61K 2039/54; A61K 2039/55511; A61K 2039/6093

(57) ABSTRACT

Some aspects of the present disclosure are generally related to injectable compositions, for example, for intratumoral drug delivery. In some embodiments, the injectable composition comprises a polymer configured to undergo a solution-gel transition when heated from room temperature to at or around body temperature, allowing for the injection of the injectable composition as a liquid with subsequent gelation to retain the composition at the injection location. In some embodiments, the injectable compositions may be capable of encapsulating radio-opaque label and/or a hydrophobic immunoadjuvant (e.g., imiquimod) at a concentration of greater than or equal to 0.5 mg/mL. Accordingly, some aspects of the present disclosure are related to the radio-opaque label facilitating visualization of the injectable composition during injection and/or the retention of the injectable composition at the injection location, allowing for localized and extended-release of the immunoadjuvant. Still other aspects are generally directed to related methods, kits, or the like.

### GOVERNMENT SPONSORSHIP

This invention was made with Government support under CA245257 awarded by the National Institutes of Health. The Government has certain rights in the invention.

### TECHNICAL FIELD

Injectable compositions, for example, for the delivery of immunoadjuvants are generally described.

### BACKGROUND

In the treatment of metastatic cancer, the abscopal effect refers to the phenomenon that a local therapy could induce therapeutic effect distantly in metastases, presumably secondary to immune activation. Intratumoral immunoadjuvants may be able to induce an abscopal rate, but existing intratumoral therapies suffer from poor local retention of the immunoadjuvant, the corresponding systemic toxicity, and the inability to confirm target delivery, leading to poor clinical trial outcomes. For example, conventionally, treatments need multiple repeated injections of the immunoadjuvant due to inaccurate delivery and leakage away from the target, combined with potentially poor aqueous solubility of the immunoadjuvant. Additionally, conventional methods lack imaging confirmation during injection, leading to missed therapeutic delivery. Local ablative therapies, such as thermal ablation may induce the abscopal effect as well, but these challenges above prevent synergy with the intratumoral immunoadjuvants. Accordingly, improved compositions for treatments and related methods are needed.

### SUMMARY

Injectable compositions for the delivery of immunoadjuvants are generally described. The subject matter of the present disclosure involves, in some cases, interrelated products, alternative solutions to a particular problem, and/or a plurality of different uses of one or more systems and/or articles.

Some aspects are related to compositions.

In some embodiments, the composition is injectable and configured for intratumoral drug delivery. In some embodiments, the injectable composition comprises a micellar structure comprising poly(lactide-co-glycolide-b-ethylene glycol-b-lactide-co-glycolide) (PLGA-PEG-PLGA) having a lactic acid (LA) to glycolic acid (GA) ratio of greater than or equal to 3:1; a hydrophobic immunoadjuvant encapsulated within the micellar structure; and a radio-opaque label present in the composition in an amount greater than or equal to 0 mg/mL and less than or equal to 200 mg/mL, wherein the injectable composition is capable of encapsulating the hydrophobic immune-adjuvant at a concentration of greater than or equal to 1 mg per 1 mL of injectable composition, wherein the molecular weight of each PLGA is greater than or equal to 500 and less than or equal to 2000, wherein the molecular weight of the PEG is greater than or equal to 500 and less than or equal to 2000, wherein the injectable composition is a liquid at room temperature and is a gel at 37° C., and wherein the immune-adjuvant exhibits an extended release profile from the injectable composition over greater than or equal to 12 hours as measured in phosphate-buffered saline at 37° C. In some embodiments, the injectable composition comprises a micellar structure comprising poly(lactide-co-glycolide-b-ethylene glycol-b-lactide-co-glycolide) (PLGA-PEG-PLGA) having a lactic acid (LA) to glycolic acid (GA) ratio of greater than or equal to 1:1; a hydrophobic immuno-adjuvant encapsulated within the micellar structure; and a radio-opaque label present in the composition in an amount greater than or equal to 0.01 mg/mL and less than or equal to 200 mg/mL, wherein the molecular weight of each PLGA is greater than or equal to 500 and less than or equal to 2000, and wherein the molecular weight of the PEG is greater than or equal to 500 and less than or equal to 2000. In some embodiments, the radio-opaque label of the composition is present in the composition in an amount greater than or equal to 0 mg/mL and less than or equal to 100 mg/mL. In some embodiments, the radio-opaque label of the composition is present in the composition in an amount greater than or equal to 15 mg/mL and less than or equal to 25 mg/mL. In some embodiments, the injectable composition is capable of encapsulating the hydrophobic immune-adjuvant at a concentration of greater than or equal to 5 mg per 1 mL of injectable composition. In some embodiments, the molecular weight of each PLGA of the injectable composition is greater than or equal to 1400 and less than or equal to 1600. In some embodiments, the molecular weight of the PEG of the injectable composition is greater than or equal to 1400 and less than or equal to 1600. In some embodiments, the immunoadjuvant of the injectable composition is a small TLR agonists. In some embodiments, the small TLR agonist of the injectable composition is selected from the group consisting of imiquimod, resiquimod, 852-A, vesitolimod, azd8848, motolimod, and selgantolimod. In some embodiments, the immunoadjuvant of the injectable composition is selected from the group comprising imiquimod, resiquimod, 852-A, vesitolimod, azd8848, motolimod, 2,3 cGAMP, NKTR-262, RG-7854, DSP-0509, BDB-001, LHC-165, BDC-1001, SHR-2150, JNJ-4964, RO-7119929, DN-1508052, VTX-1463, BNT-411, APR-003, PF-4878691, GSK-2245035, RG-7795 (ANA 773, RO 6864018), Epitirimod (R-851), DSP-3025 (AZD-8848), Sotirimod (R-850, S-30594), Telratolimod (3M-052, MEDI-9197), Isatoribine (ANA-245), Loxoribine, ANA-971, ANA-975, RG-7863 (RO6870868), and selgantolimod. In some embodiments, the injectable composition encapsulates the hydrophobic immunoadjuvant at a concentration of greater than or equal to 1 mg per 1 mL of injectable composition. In some embodiments, the injectable composition encapsulates the hydrophobic immunoadjuvant at a concentration of greater than or equal to 1 mg per 1 mL of injectable composition. In some embodiments, the micellar structure of the injectable composition comprises poly(lactide-co-glycolide-b-ethylene glycol-b-lactide-co-glycolide) (PLGA-PEG-PLGA) and has a lactic acid (LA) to glycolic acid (GA) ratio of less than or equal to 5:1. In some embodiments, the micellar structure of the injectable composition comprises poly(lactide-co-glycolide-b-ethylene glycol-b-lactide-co-glycolide) (PLGA-PEG-PLGA) and has a lactic acid (LA) to glycolic acid (GA) ratio of less than or equal to 2:1.

Some aspects are related to methods/

In some embodiments, the method is for treating a tumor. In some embodiments, the method of treating a tumor comprises injecting a composition intratumorally, wherein the composition is a liquid at room temperature and is a gel at 37° C., wherein the composition comprises: a micellar structure comprising poly(lactide-co-glycolide-b-ethylene glycol-b-lactide-co-glycolide) (PLGA-PEG-PLGA) having a lactic acid (LA) to glycolic acid (GA) ratio of greater than or equal to 3:1; and an immuno-adjuvant encapsulated within the micellar structure and present within the injectable composition at a concentration of greater than or equal to 1 mg/mL, determining the position of the composition during injection using CT, wherein the immune-adjuvant exhibits an extended release profile from the injectable composition over greater than or equal to 24 hours under physiological conditions. In some embodiments, the method further comprises performing a cryoablation concurrently to the injecting. In some embodiments, the method further comprises gelling the composition at a location of the injecting. In some embodiments, the method further comprises retaining the composition at a location of the injecting. In some embodiments, the injecting of the method occurs at a location internal to a subject. In some embodiments, the method further comprises degrading the composition at a location internal to a subject.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A is a schematic diagram of an injectable composition, according to one set of embodiments;

FIG. 1B is a schematic diagram of an injectable composition, according to one set of embodiments;

FIG. 1C is an example of the abscopal effect following percutaneous lung cryoablation (a physical immunostimulatory technique) leading to sustained reduction in tumors of hilum and portal lymphadenopathy, according to one set of embodiments;

FIG. 1D depicts locations and types of cancers amenable to local image guided drug delivery, according to one set of embodiments;

FIG. 1E depicts the treatment paradigm of CT-guided intratumoral immunogel, with or without cryoablation, in presence of systemic checkpoint inhibition immunotherapy leading to metastases regression, where the local immunostimulation induces local immune cells to release cytokines and recruit dendritic cells to sample these tumor antigens and T-cells are activated by the antigen presenting cells which then induce cancer cell death at distant sites (i.e., the “abscopal effect”), according to one set of embodiments;

FIG. 2A shows the chemical structure of hydrophilic and hydrophobic portions of the PLGA-PEG-PLGA copolymer, where LA:GA ratios can be tuned to alter gelling behavior and release, according to one set of embodiments;

FIG. 2B is a plot of the solubility of imiquimod is significantly higher encapsulated in PLGA-PEG-PLGA (LA:GA=1:1, 3:1 and 5:1 shown here) than in 10% EtOH/DI, 10% DMSO/DI, PBS, or DI water, according to one set of embodiments;

FIG. 2C is a schematic of micellar formation to enable imiquimod solubility and release, according to one set of embodiments;

FIG. 2D shows the phase transition of the gel at different temperatures centered around body temperature for LA:GA ratio of 3:1, according to one set of embodiments;

FIG. 2E shows the quantification of viscosity change over temperature as a function of LA:GA ratios, 1:1, 3:1, and 5:1, N=3, according to one set of embodiments;

FIG. 2F is a plot of dynamic light scattering measurements of micellar nanoparticle size with difference in LA:GA ratio and temperature of Imigel loaded with 6 mg/mL of imiquimod, Z-avg N=3, according to one set of embodiments;

FIG. 2G is a plot of the imiquimod release (HPLC-UV data) into PBS over time at different LA:GA ratios, 1:1, 2:1, 3:1, and 5:1, N=4, according to one set of embodiments;

FIG. 2H shows the release rate of imiquimod from an injectable composition as a function the initial concentration of imiquimod in the composition, according to one set of embodiments;

FIG. 2I shows multiple different polymer types aiming to obtain a five-day controlled release, according to one set of embodiments, where a circle is polyacrylamide; triangle is hyaluronic acid; square is PLGA-PEG-PLGA with an LA/GA ratio of 1:1; on end red square is PLGA-PEG-PLGA with an LA/GA ration of 3:1, according to one set of embodiments;

FIG. 3A shows PLGA-PEG-PLGA schematic as a function of hydrophilic and hydrophobic components, according to one set of embodiments;

FIG. 3B is a schematic of hydrophilic and hydrophobic interactions leading to liquid versus hydrogel, according to one set of embodiments;

FIG. 3C is a schematic of drug loading of imiquimod, according to one set of embodiments;

FIG. 3D is the putative release mechanism and tunability properties, according to one set of embodiments;

FIG. 3E is a synthesis schematic of loading the polymer hydrogel for injection, according to one set of embodiments;

FIG. 4A is an NMR showing the LA:GA ratio of 3:1, according to one set of embodiments;

FIG. 4B is a calculation of molecular weight and LA:GA ratio from the NMR in FIG. 4A, according to one set of embodiments;

FIG. 4C is a plot of the GPC of the polymer, according to one set of embodiments;

FIG. 5 is a plot of the relative release rate of imiquimod at different pH buffers 5.0, 6.8, and 7.4, according to one set of embodiments;

FIG. 6A is an evaluation of injectability, force of extrusion of certain volume of Imigel through a 22-gauge 15 cm needle at 20° C. (room temperature) and 37° C. as compared to normal saline and a literature-based maximum hand injection force of 50 N, n=3, according to one set of embodiments;

FIG. 6B is a visualization of the injected Imigel containing 100 mg mL−1 Iopamidol contrast agent into a dorsal flank tumor (right) versus naïve tumor (left), according to one set of embodiments;

FIG. 6C shows H&E staining demonstrating cleft in the tumor created by gel injection (arrow), where it shows a representative image of n=3 and the scale represents 200 μm, according to one set of embodiments;

FIG. 6D shows the release profile of imiquimod from micellar structures comprising either PLGA1100-PEG1000-PLGA1100 or PLGA1600-PLGA1500-PLGA1600 the in the absence and presence of iopamidol at 5 mg/ml, according to one set of embodiments;

FIG. 6E shows the release profile as a function of LA/GA ratios, as well as in the absence and presence of iopamidol when the LA/GA ratio is 3:1, according to one set of embodiments;

FIG. 7A shows a survival study demonstrating the abscopal effect of Imigel in immunotherapy-resistant dual tumor models with a murine experimental model in dual tumor balb/c mice receiving concomitant intraperitoneal (systemic) injection of checkpoint inhibition (CPI), on days 3, 6, and 9, where on Day 8, tumors measuring at least 6 mm were randomized into treatment categories, according to one set of embodiments;

FIG. 7B shows, in a CT-26 cancer model, a single Imigel depot achieved similar survival as 6 daily injections, with or without the use of cryoablation, n=8-18 mice per arm, according to one set of embodiments;

FIG. 7C, shows the CPI-resistant EMT-6 breast cancer model showed synergistic enhancement in survival when Imigel was used where cryoablation only did not lead to survival, n=8-19 mice per arm and all animals that survived to 90 days had full regression of the ipsilateral treated and contralateral untreated tumors demonstrating the abscopal effect and where all animals that did not survive had at least one tumor that grew to 2 cm and were euthanized, according to one set of embodiments;

FIG. 8A shows tumor growth curves for ipsilateral (treated) and contralateral (untreated, metastasis model) tumors, comparing systemic checkpoint inhibition (CPI) immunotherapy with CPI+Imigel, split into responders and non-responders, according to one set of embodiments;

FIG. 8B shows flow cytometry results, showing unchanged CD8 T cells in ipsilateral and contralateral tumors, while demonstrating a significant change in number of CD44+/PD1+CD8 T cells with Imigel treatment compared to CPI alone (p=0.01), and also with CPI+6× daily imiquimod (p=0.04) where systemic CPI alone are the same data points in both contralateral and ipsilateral, given there is no specifically treated tumor, according to one set of embodiments;

FIG. 8C shows granzyme B immunofluorescence (IF) and DAPI staining of tumors, and H&E images of the studied regions, for the different cohorts as in (FIG. 8B), according to one set of embodiments;

FIG. 8D shows cytokine analysis of ipsilateral and contralateral CT-26 tumors, compared to serial imiquimod in absolute fold change, according to one set of embodiments;

FIGS. 9A-9J show intratumoral therapies, imigel or cryoablation, Ipsilateral refers to side treated by intervention intratumorally via imigel, or cryoablation, and contralateral refers to the untreated tumor, where each tumor growth curve is shown as compared to checkpoint inhibitor (CPI) tumor growth alone (N=8, red curves for CT-26 tumor models, and N=15 for EMT-6 tumor models), where, for systemic checkpoint inhibitors treatment only mice, ipsilateral was defined as the left tumor and contralateral as the right tumor, where Imigel refers to 300 ug imiquimod+PLGA-PEG-PLGA (1500:1500:1500, LA:GA 3:1), where comparison groups are as follows: A) Ipsilateral tumor growth+CPI (N=15) in CT-26 dual tumor models B) Contralateral tumors of imigel+CPI (N=15) therapy arm in CT-26 dual tumor models, ipsilateral side C) Ipsilateral tumors of Cryoablation+Imigel+CPI (N=18) therapy arm of CT 26 model D) Contralateral tumors of Cryoablation+Imigel+CPI (N=18) therapy arm of CT 26 model E) Contralateral tumor growth for the therapy arm of daily low dose imiquimod injections (50 ug each day×6 days) N=13, F) Contralateral side cryoablation, daily low dose imiquimod injections (50 ug each day×6 days), and checkpoint inhibitor therapy N=14. G) Ipsilateral tumor growth in CPI+Imigel therapy arm EMT-6 tumor model (N=8) H) Contralateral tumor growth in CPI+Imigel therapy arm EMT-6 tumor model (N=8), I) Ipsilateral tumor growth in CPI+Cryoablation+Imigel therapy arm EMT-6 tumor model (N=18), J) Contralateral tumor growth in CPI+Cryoablation+Imigel therapy arm EMT-6 tumor model (N=18), according to one set of embodiments;

FIG. 10A is a plot of viscosity vs. shear rate of an injectable composition at different temperatures, according to one set of embodiments;

FIG. 10B is a plot of the storage modulus and loss modulus of an injectable composition as a function of angular frequency at different temperatures, according to one set of embodiments;

FIG. 11 shows the release profile of motolimod as a function of time, according to one set of embodiments;

FIG. 12A show CT scans pre-and-post injection of an injectable composition in the presence of barium, iopamidol (Isovue), and lipiodol, according to one set of embodiments;

FIG. 12B shows a release profile of imiquimod in phosphate buffer solution from an injectable composition in the absence and presence of barium sulfate in the composition; and

FIG. 13 is a plot of gel permeation chromatography (GPC) retention of various injectable compositions, according to one set of embodiments.

### DETAILED DESCRIPTION

Some aspects of the present disclosure are generally related to injectable compositions, for example, for intratumoral drug delivery. In some embodiments, the injectable composition comprises a polymer configured to undergo a solution-gel transition when heated from room temperature to at or around body temperature, allowing for the injection of the injectable composition as a liquid with subsequent gelation to retain the composition at the injection location. In some embodiments, the injectable compositions may be capable of encapsulating radio-opaque label and/or a hydrophobic immunoadjuvant (e.g., imiquimod) at a concentration of greater than or equal to 0.5 mg/mL. Accordingly, some aspects of the present disclosure are related to the radio-opaque label facilitating visualization of the injectable composition during injection and/or the retention of the injectable composition at the injection location, allowing for localized and extended-release of the immunoadjuvant. Still other aspects are generally directed to methods of making, using, and/or administering the compositions, kits containing the compositions, or the like.

Percutaneous cryoablation is a common clinical therapy for metastatic and primary cancer. There are clinical reports of cryoablation inducing regression of distant metastases, known as the abscopal effect. Abscopal refers to the phenomenon that a local therapy could induce therapeutic effect distantly in metastases, presumably secondary to immune activation. Intratumoral immunoadjuvants may be able to augment the abscopal rate of cryoablation, but existing intratumoral therapies suffer from poor aqueous solubility of the immunoadjuvant, the corresponding need for frequent injections, and the inability to confirm target delivery, leading to poor clinical trial outcomes. For example, conventionally, treatments need multiple repeated injections of the immunoadjuvant due to the poor aqueous solubility of the immunoadjuvant and accordingly small delivery payloads during injections. Additionally, conventional methods lack imaging confirmation during injection. That is, the injection of visceral organs or deep lymph nodes by interventional radiologists generally requires CT- or ultrasound guidance and procedural sedation. Without some imaging agent embedded, it is challenging to verify the targeted delivery to delivery or assess for off-target delivery during or after the procedure-which is critical for the evaluation of therapeutic efficacy and therapeutic window.

In view of the above, the inventors have recognized and developed an injectable, thermoresponsive, locally resident and imagable viscous gel to contain and deliver a high concentration of immunoadjuvants (e.g., imiquimod). In some embodiments, the injection of the composition is configured for concurrent delivery with cryoablation to increase the resulting immune response in the subject. The term “subject,” as used herein, refers to an individual organism such as a human or an animal. In some embodiments, the subject is a mammal (e.g., a human, a non-human primate, or a non-human mammal), a vertebrate, a laboratory animal, a domesticated animal, an agricultural animal, or a companion animal. In some embodiments, the subject is a human. In some embodiments, the subject is a rodent, a mouse, a rat, a hamster, a rabbit, a dog, a cat, a cow, a goat, a sheep, or a pig.

Accordingly, some aspects of the present disclosure are related to injectable compositions, for example, that are configured for drug delivery. In some such embodiments, the injectable compositions are configured for intratumoral drug delivery.

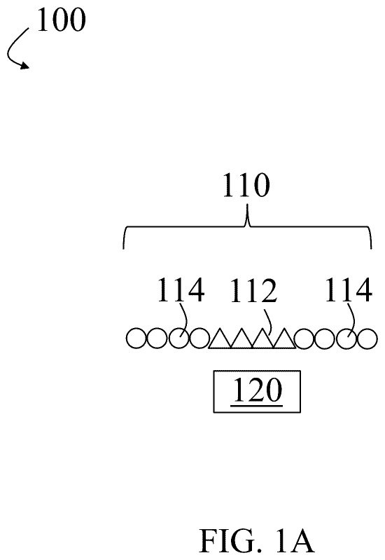

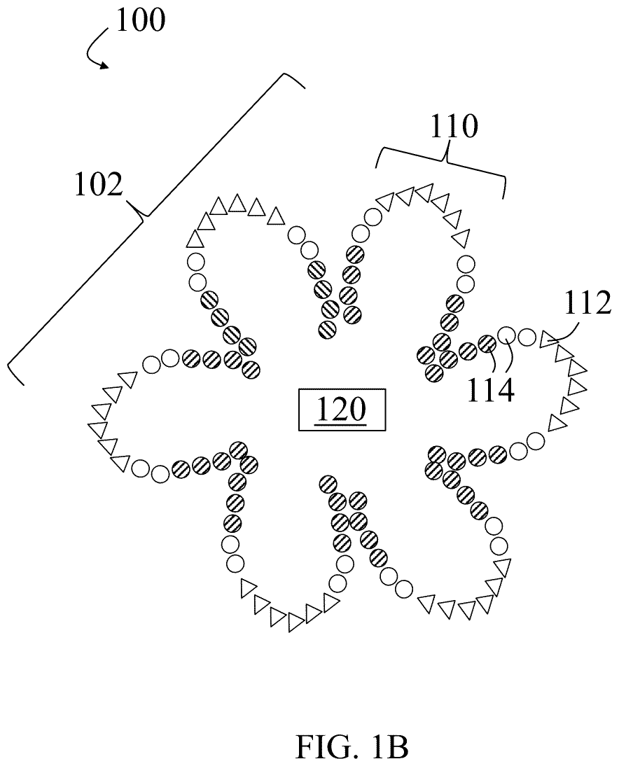

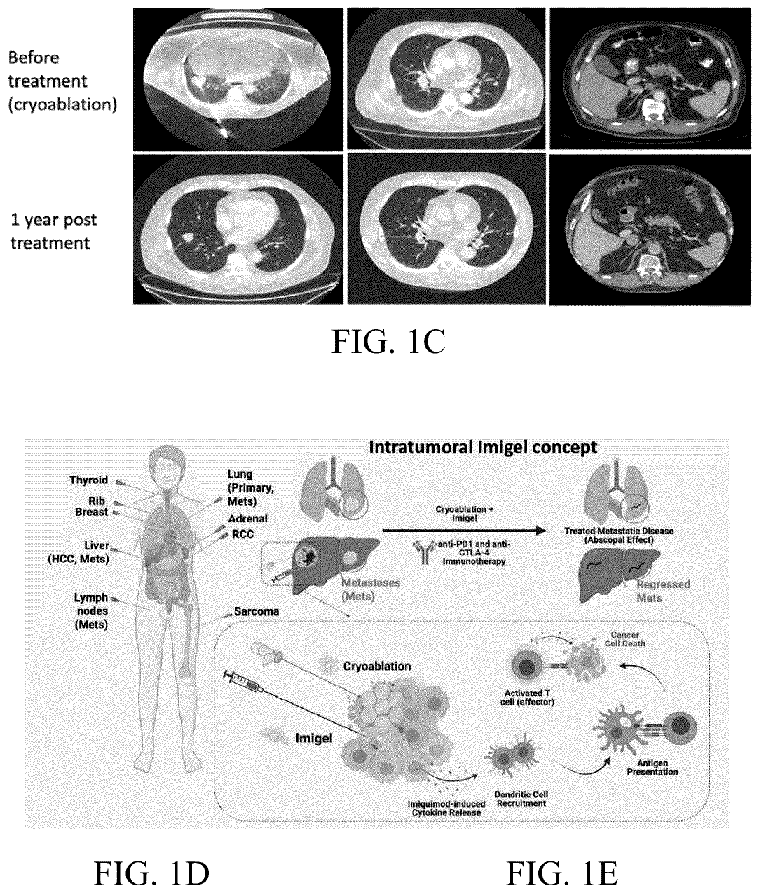

In some embodiments, as illustrated in FIG. 1A, injectable composition 100 comprises block copolymer 110 and one or more active substances 120 (e.g., a hydrophobic immunoadjuvant). In some embodiments, block copolymer 110 comprises poly(lactide-co-glycolide-b-ethylene glycol-b-lactide-co-glycolide) (PLGA-PEG-PLGA). For example, in some embodiments, block copolymer 110 comprises PEG chain 112 and PLGA chains 114 and 116. Each PEG chain may be the same or different (e.g., having different molecular weights), optionally substituted. Each PLGA chain may be the same or different (e.g., having different molecular weights), and/or optionally substituted.

In some embodiments, active substance 120 is associated with block copolymer 110 (e.g., via hydrophobic interactions, hydrostatic interactions, via a bond, etc.). In some embodiments, the association is by thermodynamically favorable association between hydrophobic groups expelling water. In some embodiments, the active substance is associated with the block copolymer via formation of a bond, such as an ionic bond, a covalent bond, a hydrogen bond, Van der Waals interactions, and/or the like. The covalent bond may be, for example, carbon-carbon, carbon-oxygen, oxygen-silicon, sulfur-sulfur, phosphorus-nitrogen, carbon-nitrogen, metal-oxygen, or other covalent bonds. The hydrogen bond may be, for example, between hydroxyl, amine, carboxyl, thiol, and/or similar functional groups. For example, in some embodiments, the block copolymer is functionalized (e.g., a PLGA group further comprises a functional group, a PEG group further comprises a functional group) such that the block copolymer is capable of forming a bond with the active substance (e.g., via a functional group). In other embodiments, the active substance is non-covalently associated with the block copolymer. In some such embodiments, the active substance may be dispersed or encapsulated within a portion of the block copolymer e.g., by hydrophilic and/or hydrophobic forces.

The injectable composition may include a micellar structure. In some embodiments, the block copolymer (e.g., PLGA-PEG-PLGA) forms a micellar structure in solution. Advantageously, in some embodiments, the micellar structure may include a substantially hydrophobic interior and substantially hydrophilic exterior such that the micellar structure may be miscible and/or soluble with aqueous solutions while the interior may be primarily hydrophobic such that hydrophobic active substances (e.g., hydrophobic immunoadjuvants) may be soluble at relatively high loadings, as described in more detail elsewhere herein.

According to some embodiments, a micellar structure of the injectable compositions comprises a block copolymer. In some such embodiments, the micellar structure comprises poly(lactide-co-glycolide-b-ethylene glycol-b-lactide-co-glycolide) (PLGA-PEG-PLGA). The lactic acid (LA) to glycolic acid (GA) ratio in the PLGA blocks of the PLGA-PEG-PLGA block copolymer, in some embodiments, may impact the micellar structure and/or how the micellar structure encapsulates active substances, as described in more detail elsewhere herein. For example, LA includes a methyl group, and is accordingly more hydrophobic when compared to GA. Thus, by increasing the content of the LA in the PLGA blocks, the PLGA may become relatively more hydrophobic when compared to PLGA blocks with relatively less LA. While much of the disclosure herein is generally related to tri-block copolymers, those of ordinary skill in the art would understand that other multi-block copolymers of combinations of PLGA and PEG (e.g., of varying molecular weight) are also possible. For example, without wishing to be limited as such, in some embodiments, the composition may comprise a multiblock copolymer having a structure as in poly(lactide-b-glycolide-b-ethylene glycol-b-lactide-b-glycolide) (e.g., PLA-PGA-PEG-PLA-PGA, where the LA and GA of the PLGA block are blocks rather than being randomly interspersed). Other non-limiting examples include PLA-PGA-PEG-PGA-PLA and PLA-PGA-PLA-PEG-PLA-PGA-PLA, and PGA-PLA-PGA-PEG-PGA-PLA-PGA. Without wishing to be bound by theory, in some embodiments, the distribution of the LA and GA within the PLGA block may also affect the thermoresponsive behavior of the injectable composition.

FIG. 1B depicts a schematic diagram of an example micellar structure of an injectable composition. In this embodiment, an injectable composition 100 comprises micellar structure 102 comprising block copolymer 110. In some embodiments, block copolymer 110 is PLGA-PEG-PLGA, configured and arranged such that the PEG blocks 112 face a hydrophilic exterior (e.g., an aqueous solution) and the PLGA blocks 114 are oriented inwards towards a relatively more hydrophobic core of the micellar structure 102. The micellar structure 102 may, in some embodiments, encapsulate one or more active substances 120. For example, in some embodiments, the injectable composition is constructed and arranged to release the active substance (e.g., hydrophobic immunoadjuvant) from the micellar structure(s) (e.g., upon delivery to a subject such as intratumoral injection). In some embodiments, the active substance is designed for release from the micellar structure. Such embodiments may be useful in the context of drug delivery. In some embodiments, the active substance may be bound (e.g., via one or more bonds such as a covalent bond, an ionic bond, etc. as described above) to the micellar structure. In other embodiments, the active substance is non-covalently associated with the micellar structure. In some such embodiments, the active substance may be dispersed or encapsulated within the micellar structure by hydrophilic and/or hydrophobic forces.

In FIG. 1B, the PLGA blocks are depicted to include a 2:1 ratio of LA to GA groups. The micellar structure comprising the PLGA-PEG-PLGA, in accordance with some embodiments, may have any of a variety of suitable LA to GA ratios. In some embodiments, the LA to GA ratio is greater than or equal to 1:1, greater than or equal to 2:1, greater than or equal to 3:1, greater than or equal to 4:1, greater than or equal to 5:1, greater than or equal to 6:1, greater than or equal to 7:1, greater than or equal to 8:1, or greater than or equal to 9:1. According to some embodiments, the LA to GA ratio is less than or equal to 10:1, less than or equal to 9:1, less than or equal to 8:1, less than or equal to 7:1, less than or equal to 6:1, less than or equal to 5:1, less than or equal to 4:1, less than or equal to 3:1, or less than or equal to 2:1. Combinations of the foregoing ranges are possible (e.g., greater than or equal to 1:1 and less than or equal to 10:1, greater than or equal to 1:1 and less than or equal to 5:1, greater than or equal to 1:1 and less than or equal to 2:1, greater than or equal to 3:1 and less than or equal to 5:1). Other ranges are also possible. In some embodiments, the LA to GA ratio in each of the PLGA blocks of the block copolymer (e.g., PLGA-PEG-PLGA) may, on average, be the same or different.

In some embodiments, the LA to GA ratio in the PLGA block of the copolymer (e.g., a PLGA-PEG-PLGA copolymer) impacts the size of micelles formed therefrom. For example, when the LA to GA ratio is relatively low (e.g., greater than or equal to 1:1 and less than or equal to 2:1), the PLGA block is relatively more hydrophilic. Accordingly, the PLGA blocks may not pack as densely (e.g., see micellar structure in FIG. 1B), resulting in relatively larger micelles with more water dispersed therein. The larger micelles may facilitate a high loading capacity of the hydrophobic immunoadjuvant. In contrast, when the LA to GA ratio is relatively high (e.g., greater than or equal to 3:1 and less than or equal to 5:1), the PLGA block is relatively more hydrophobic. In some such cases, the PLGA blocks may pack more densely leading to smaller micelles. In some embodiments, the smaller micelles have a smaller loading capacity of the hydrophobic immunoadjuvant than the larger micelles.

As described above, a micellar structure comprising PLGA-PEG-PLGA may be more or less densely packed based on the LA to GA ratio of the PLGA blocks, in accordance with some embodiments. Accordingly, in some embodiments, an average maximum dimension of the micellar structures in the injectable composition may be determined using dynamic light scattering. In some embodiments, an average maximum dimension of the micellar structures in the injectable composition is greater than or equal to 50 nm, greater than or equal to 60 nm, greater than or equal to 70 nm, greater than or equal to 80 nm, greater than or equal to 90 nm, greater than or equal to 100 nm, greater than or equal to 110 nm, greater than or equal to 120 nm, greater than or equal to 130 nm, greater than or equal to 140 nm, greater than or equal to 150 nm, greater than or equal to 160 nm, greater than or equal to 170 nm, greater than or equal to 180 nm, or greater than or equal to 190 nm. According to some embodiments, the average maximum dimension of the micellar structures in the injectable composition is less than or equal to 200 nm, less than or equal to 190 nm, less than or equal to 180 nm, less than or equal to 170 nm, less than or equal to 160 nm, less than or equal to 150 nm, less than or equal to 140 nm, less than or equal to 130 nm, less than or equal to 120 nm, less than or equal to 110 nm, less than or equal to 100 nm, less than or equal to 90 nm, less than or equal to 80 nm, or less than or equal to 70 nm. Combinations of the foregoing ranges are possible (e.g., greater than or equal to 50 nm and less than or equal to 200 nm, greater than or equal to 70 nm and less than or equal to 120 nm, greater than or equal to 70 nm and less than or equal to 90 nm). Other ranges are also possible.

In some embodiments, the molecular weight (e.g., size) of each block of the block copolymer may affect the resulting structure of the micelles. In some embodiments, the molecular weight (e.g., number average molecular weight) of each PLGA block present in a PLGA-PEG-PLGA copolymer of the injectable composition is independently greater than or equal to 500, greater than or equal to 600, greater than or equal to 700, greater than or equal to 800, greater than or equal to 900, greater than or equal to 1000, greater than or equal to 1100, greater than or equal to 1200, greater than or equal to 1300, greater than or equal to 1400, greater than or equal to 1500, greater than or equal to 1600, greater than or equal to 1700, greater than or equal to 1800, or greater than or equal to 1900. In some embodiments, the molecular weight (e.g., number average molecular weight) of each PLGA block present in a PLGA-PEG-PLGA copolymer of the injectable composition is independently less than or equal to 2000, less than or equal to 1900, less than or equal to 1800, less than or equal to 1700, less than or equal to 1600, less than or equal to 1500, less than or equal to 1400, less than or equal to 1300, less than or equal to 1200, less than or equal to 1100, less than or equal to 1000, less than or equal to 900, less than or equal to 800, less than or equal to 700, or less than or equal to 600. Combinations of the foregoing ranges are possible (e.g., greater than or equal to 500 and less than or equal to 2000, greater than or equal to 1400 and less than or equal to 1600). Other ranges are also possible. Note that, in some embodiments, the molecular weight of each PLGA block is, on average, the same. In other embodiments, the molecular weight of each PLGA block is different. For example, a first PLGA of the PLGA-PEG-PLGA copolymer may have a first molecular weight of greater than or equal to 500 and less than or equal to 2000 (e.g., greater than or equal to 1400 and less than or equal to 1600) and a second PLGA of the PLGA-PEG-PLGA copolymer may have a second molecular weight of greater than or equal to 500 and less than or equal to 2000 (e.g., greater than or equal to 1400 and less than or equal to 1600), different than the first molecular weight.

In some embodiments, the molecular weight (e.g., number average molecular weight) of each PEG present in a PLGA-PEG-PLGA copolymer of the injectable composition is greater than or equal to 500, greater than or equal to 600, greater than or equal to 700, greater than or equal to 800, greater than or equal to 900, greater than or equal to 1000, greater than or equal to 1100, greater than or equal to 1200, greater than or equal to 1300, greater than or equal to 1400, greater than or equal to 1500, greater than or equal to 1600, greater than or equal to 1700, greater than or equal to 1800, or greater than or equal to 1900. In some embodiments, the molecular weight (e.g., number average molecular weight) of the PEG present in a PLGA-PEG-PLGA copolymer of the injectable composition is less than or equal to 2000, less than or equal to 1900, less than or equal to 1800, less than or equal to 1700, less than or equal to 1600, less than or equal to 1500, less than or equal to 1400, less than or equal to 1300, less than or equal to 1200, less than or equal to 1100, less than or equal to 1000, less than or equal to 900, less than or equal to 800, less than or equal to 700, or less than or equal to 600. Combinations of the foregoing ranges are possible (e.g., greater than or equal to 500 and less than or equal to 2000, greater than or equal to 1400 and less than or equal to 1600). Other ranges are also possible.

As described above, in some embodiments, the injectable composition may be configured to be a liquid at room temperature and to be a gel at body temperature. Advantageously, in some embodiments, the injectable composition remains a liquid at or around room temperature to facilitate injection. In some embodiments, upon heating, the injectable composition may undergo a solution-gelation transition (e.g., a sol-gel transition). Advantageously, in accordance with some embodiments, the sol-gel transition may occur at or around body temperature such that, following injection of the liquid injectable composition at a location internal to the subject, the solution may heat and gel to remain at the location of injection. Maintaining a location of the injectable composition via gelation, in some embodiments, may facilitate a localized extended release of a hydrophobic immunoadjuvant of the injectable composition to the localized region within the subject. In some embodiments, a LA to GA ratio of greater than or equal to 1:1 and less than or equal to 10:1 may facilitate a sol-gel transition at such temperatures. In some embodiments, a LA to GA ratio of greater than or equal to 3:1 and less than or equal to 5:1 may facilitate a sol-gel transition at such temperatures. As noted elsewhere herein, in some embodiments, the thermoresponsive behavior (e.g., the sol-gel temperature) may also be affected by the distribution of the LA and GA within the PLGA blocks.

In some embodiments, the injectable composition remains a liquid at a temperature of greater than or equal to 18° C., greater than or equal to 19° C., greater than or equal to 20° C., greater than or equal to 21° C., greater than or equal to 22° C., greater than or equal to 23° C., greater than or equal to 24° C., greater than or equal to 25° C., greater than or equal to 26° C., greater than or equal to 27° C., greater than or equal to 28° C., or greater than or equal to 29° C. In some embodiments, the injectable composition remains a liquid at a temperature of less than or equal to 30° C., less than or equal to 29° C., less than or equal to 28° C., less than or equal to 27° C., less than or equal to 26° C., less than or equal to 25° C., less than or equal to 24° C., less than or equal to 23° C., less than or equal to 22° C., less than or equal to 21° C., less than or equal to 20° C., or less than or equal to 19° C. Combinations of the foregoing ranges are possible (e.g., greater than or equal to 18° C. and less than or equal to 30° C., greater than or equal to 20° C. and less than or equal to 25° C.). Other ranges are also possible, and may be dependent on the components of the injectable composition.

In some embodiments, the injectable composition may undergo a sol-gel transition at or around body temperature of a subject. In some embodiments, the sol-gel transition may at least partially occur at a temperature of greater than or equal to 35° C., greater than or equal to 36° C., greater than or equal to 37° C., greater than or equal to 38° C., or greater than or equal to 39° C. In some embodiments, the sol-gel transition may at least partially occur at a temperature of less than or equal to 40° C., less than or equal to 39° C., less than or equal to 38° C., less than or equal to 37° C., or less than or equal to 36° C. Combinations of the foregoing ranges are possible (e.g., greater than or equal to 35° C. and less than or equal to 40° C., greater than or equal to 36° C. and less than or equal to 37° C.). Other ranges are also possible.

Still, in some embodiments, the LA to GA ratio may be selected such that the PLGA-PEG-PLGA solution does not gel and, due to the interior of the micellar structures including a larger proportion of hydrophilic GA residues, burst release of the hydrophobic immunoadjuvant from the micellar structure following injection may occur. In some embodiments, burst release may be chosen when, for example, extended release of the compounds contained within the micellar structure provides no benefits over burst release. To achieve a burst release, in some embodiments, a relatively lower LA to GA ratio may be chosen (e.g., greater than or equal to 1:1 and less than or equal to 2:1).

In some embodiments, the injectable composition comprises a hydrophobic immunoadjuvant. In some embodiments, the hydrophobic immunoadjuvant comprises a small molecule having a molecular weight of less than about 2500 Daltons, less than about 2000 Daltons, less than about 1500 Daltons, less than about 1000 Daltons, less than about 750 Daltons, less than about 500 Daltons, less or than about 400 Daltons. In some cases, the hydrophobic immunoadjuvant is a small molecule having a molecular weight between 200 Daltons and 400 Daltons, between 400 Daltons and 1000 Daltons, or between 500 Daltons and 2500 Daltons.

The hydrophobic immunoadjuvant may be any of a variety of small molecules, according to some embodiments. Non-limiting examples of suitable hydrophobic immunoadjuvants include imidazoquinoline derivatives and immunoadjuvant drug imiquimod (e.g., TLR 7 agonist), Motolimod (TLR 8 agonist), Vesatolimod (TLR 7 agonist), 852-A, azd8848, Selgantolimod, resiquimod, and 2,3 cGAMP, and derivatives thereof. In some embodiments, the hydrophobic immunoadjuvant may be a small toll-like receptor (TLR) agonist. In some such embodiments, the small TLR agonist is selected from the group consisting of imiquimod, resiquimod, 852-A, vesitolimod, azd8848, motolimod, and selgantolimod, and derivatives thereof. Other hydrophobic immunoadjuvants are also possible.

Additional non-limiting examples of suitable immunoadjuvants include NKTR-262, RG-7854, DSP-0509, BDB-001, LHC-165, BDC-1001, SHR-2150, JNJ-4964, RO-7119929, DN-1508052, VTX-1463, BNT-411, APR-003, PF-4878691, GSK-2245035, RG-7795 (ANA 773, RO 6864018), Epitirimod (R-851), DSP-3025 (AZD-8848), Sotirimod (R-850, S-30594), Telratolimod (3M-052, MEDI-9197), Isatoribine (ANA-245), Loxoribine, ANA-971, ANA-975, and RG-7863 (RO6870868).

According to some embodiments, as described above, the injectable composition includes a micellar structure comprising PLGA-PEG-PLGA. Advantageously, in some embodiments, the injectable compositions described herein may be useful as a general platform for delivery of a wide variety of hydrophobic immunoadjuvants that may not be typically delivered via injection due to their poor aqueous solubility. In some embodiments, the PLGA-PEG-PLGA micellar structure is capable of encapsulating a hydrophobic immunoadjuvant in high concentrations. For example, as illustrated in FIG. 1B, one or more active substances 120 (e.g., a hydrophobic immunoadjuvant) may be encapsulated within a micellar structure of the injectable composition. Encapsulating the immunoadjuvant within the micellar structures of the injectable composition may be desirable, in some embodiments, to facilitate the solubilization and/or delivery of the immunoadjuvant in high concentrations to a location internal of a subject that may otherwise not be possible.

For instance, in some embodiments, the PLGA components of a PLGA-PEG-PLGA micellar structure forms a relatively hydrophobic region when compared to an aqueous solution, e.g., indiscriminate of the LA to GA ratio present in the PLGA blocks. In some embodiments, the hydrophobic nature of the PLGA block (e.g., compared to an aqueous solution) facilitates a relatively high solubility and thus loading of a hydrophobic immunoadjuvant into the micellar structures of the injectable compositions when compared to the aqueous solubility of the immunoadjuvant.

For instance, in some embodiments, an immunoadjuvant may be hydrophobic and thus may not be substantially soluble in aqueous solutions (e.g., a hydrophobic immunoadjuvant). In some embodiments, a hydrophobic immunoadjuvant may have a solubility in an aqueous solution of less than or equal to 0.1 mg/mL, less than or equal to 0.05 mg/mL, less than or equal to 0.01 mg/mL, less than or equal to 0.005 mg/mL, or less than or equal to 0.002 mg/mL. In contrast, the solubility of the hydrophobic immunoadjuvant may be relatively high in the micellar structures described herein. For example, the micellar structure may be capable of encapsulating, may be configured to encapsulate, and/or may encapsulate the immunoadjuvant in a high concentration.

According to some embodiments, the micellar structure may be capable of encapsulating, may be configured to encapsulate, and/or may encapsulate the hydrophobic immunoadjuvant in a concentration of greater than or equal to 0.1 mg/mL (e.g., 0.1 mg immunoadjuvant per mL of injectable composition), greater than or equal to 0.5 mg/mL, greater than or equal to 1 mg/mL, greater than or equal to 2 mg/mL, greater than or equal to 3 mg/mL, greater than or equal to 4 mg/mL, greater than or equal to 5 mg/mL, greater than or equal to 6 mg/mL, or greater than or equal to 7 mg/mL. In some embodiments, the micellar structure may be capable of encapsulating, may be configured to encapsulate, and/or may encapsulate the hydrophobic immunoadjuvant in a concentration of less than or equal to 8 mg/mL, less than or equal to 7 mg/mL, less than or equal to 6 mg/mL, less than or equal to 5 mg/mL, less than or equal to 4 mg/mL, less than or equal to 3 mg/mL, less than or equal to 2 mg/mL, less than or equal to 1 mg/mL, or less than or equal to 0.5 mg/mL. Combinations of the foregoing ranges are possible (e.g., greater than or equal to 0.1 mg/mL and less than or equal to 8 mg/mL, greater than or equal to 5 mg/mL and less than or equal to 6 mg/mL). Following encapsulation of the hydrophobic immunoadjuvant by the micellar structures, the micellar structures may then be delivered to a subject to deliver the immunoadjuvants container therein.

In some embodiments, high concentrations of the hydrophobic immunoadjuvant in the injectable composition may be desirable. However, in other embodiments, a low concentration of the hydrophobic immunoadjuvant in the injectable composition may be desirable if the immunoadjuvant has a high activity.

According to some embodiments, the immunoadjuvant may further comprise a radio-opaque label. In some embodiments, a radio-opaque label may be encapsulated within a micellar structure of the injectable composition and, in some cases, may be co-encapsulated along with a hydrophobic immunoadjuvant. The presence of the radio-opaque label may facilitate the visualization and thus targeted injection of the injectable composition, e.g., by injecting the injectable composition containing the radio-opaque label during a computer tomography (CT) scan. Note, however, that the presence of both the radio-opaque label and the hydrophobic immunoadjuvant, in some embodiments, may deleteriously impact the ability of the injectable composition to retain either the radio-opaque label or the hydrophobic immunoadjuvant, which may result in a burst release of the radio-opaque label and the hydrophobic immunoadjuvant upon injection of the injectable composition. In some such embodiments, burst release may occur in the present of both the radio-opaque label and the hydrophobic immunoadjuvant without regard to the composition of the block polymer of the injectable gel.

The radio-opaque label may be any of a variety of agents, in accordance with some embodiments. Not limiting examples of radio-opaque labels include iopamidol, calcium, calcium chloride, barium, barium sulfate, zinc, gold, titanocene, iohexol, diatrizoate meglumine, diatrizoate sodium, ethiodized oil, and gadolinium. Other labels are also possible.

Accordingly, in some embodiments, the radio-opaque label may be present in the injectable composition in a concentration sufficient to allow for imaging of the injectable composition during injection, e.g., by a CT scan or ultrasound, but not in too high of a concentration to result in a burst release of the label and/or the hydrophobic immunoadjuvant from the micellar structures. In some embodiments, the radio-opaque label may be present in the injectable composition in an amount of greater than 0 mg/mL (e.g., mg of the radio-opaque label per mL of the injectable composition), greater than or equal to 0.1 mg/mL, greater than or equal to 0.5 mg/mL, greater than or equal to 1 mg/mL, greater than or equal to 2 mg/mL, greater than or equal to 3 mg/mL, greater than or equal to 5 mg/mL, greater than or equal to 8 mg/mL, greater than or equal to 10 mg/mL, greater than or equal to 15 mg/mL, greater than or equal to 18 mg/mL, greater than or equal to 20 mg/mL, greater than or equal to 22 mg/mL, greater than or equal to 25 mg/mL, greater than or equal to 28 mg/mL, greater than or equal to 35 mg/mL, greater than or equal to 50 mg/mL, greater than or equal to 75 mg/mL, greater than or equal to 90 mg/mL, greater than or equal to 100 mg/mL, or greater than or equal to 150 mg/mL. In some embodiments, the radio-opaque label may be present in the injectable composition in an amount of less than or equal to 200 mg/mL, less than or equal to 150 mg/mL, less than or equal to 100 mg/mL, less than or equal to 90 mg/mL, less than or equal to 75 mg/mL, less than or equal to 50 mg/mL, less than or equal to 35 mg/mL, less than or equal to 28 mg/mL, less than or equal to 25 mg/mL, less than or equal to 22 mg/mL, less than or equal to 20 mg/mL, less than or equal to 18 mg/mL, less than or equal to 15 mg/mL, less than or equal to 10 mg/mL, less than or equal to 8 mg/mL, less than or equal to 5 mg/mL, less than or equal to 3 mg/mL, less than or equal to 2 mg/mL, less than or equal to 1 mg/mL, less than or equal to 0.5 mg/mL, or less than or equal to 0.1 mg/mL. Combinations of the foregoing ranges are possible (e.g., greater than 0 and less than or equal to 200 mg/mL, greater than or equal to 15 mg/mL and less than or equal to 28 mg/mL). Other ranges are also possible.

As described above, in some embodiments, the injectable compositions may be configured to be a liquid at room temperature and gel at or around body temperature. In some embodiments, due to the gelation, the injectable composition may remain at the location of injection for a relatively long time, for example, until injectable composition degrades. For instance, in some embodiments where the injectable composition includes a micellar structure comprising a polymer, the polymer may be biodegradable and may degrade over the course of greater than or equal to 1 hours, greater than or equal to 10 hours, greater than or equal to 1 day, greater than or equal to 2 days, greater than or equal to 3 days, greater than or equal to 4 days, greater than or equal to 5 days, greater than or equal to 6 days, or greater than or equal to 7 days. In some embodiments, the polymer of the micellar structure of the injectable composition may be biodegradable and may degrade over the course of less than or equal to 8 days, less than or equal to 7 days, less than or equal to 6 days, less than or equal to 5 days, less than or equal to 4 days, less than or equal to 3 days, less than or equal to 2 days, less than or equal to 1 day, or less than or equal to 10 hours. Combinations of the foregoing ranges are possible (e.g., greater than or equal to 1 hour and less than or equal to 8 days). Other ranges are also possible.

In some embodiments, a micellar structure of the injectable composition may encapsulate a drug, e.g., a hydrophobic immunoadjuvant. In some such embodiments, due to the hydrophobic nature of the interior of the micellar structure (e.g., when the LA to GA ratio is greater than or equal to 1:1 and less than or equal to 10:1, when the LA to GA ratio is greater than or equal to 3:1 and less than or equal to 5:1), the hydrophobic immunoadjuvant may have relatively slow release kinetics from the interior of the micellar structure to the exterior of the micellar structure (e.g., into the subject). For instance, initially, the hydrophobic immunoadjuvant may only release from the micellar structure by diffusion. In some embodiments, the release rate of the hydrophobic immunoadjuvant may increase over time due to the degradation of the micellar structure, for instance, if the structure is biodegradable.

Due to the micellar structure retaining the hydrophobic immunoadjuvant, in some embodiments, the time over which the immunoadjuvant is released from the micellar structure may be relatively long. In some embodiments, the immunoadjuvant exhibits an extended release profile from the injectable composition as measured in phosphate-buffered saline at 37° C. For example, in some embodiments, the time over which at least 90 wt % of the immunoadjuvant is released from the micellar structure is greater than or equal to 24 hours, greater than or equal to 2 days, greater than or equal to 3 days, greater than or equal to 4 days, greater than or equal to 5 days, greater than or equal to 6 days, greater than or equal to 7 days, greater than or equal to 8 days, greater than or equal to 9 days, greater than or equal to 10 days, greater than or equal to 12 days, greater than or equal to 15 days, greater than or equal to 18 days, greater than or equal to 20 days, or greater than or equal to 25 days. In some embodiments, the time over which at least 90 wt % of the immunoadjuvant is released from the micellar structure is less than or equal to 30 days, less than or equal to 25 days, less than or equal to 20 days, less than or equal to 18 days, less than or equal to 15 days, less than or equal to 12 days, less than or equal to 10 days, less than or equal to 9 days, less than or equal to 8 days, less than or equal to 7 days, less than or equal to 6 days, less than or equal to 5 days, less than or equal to 4 days, less than or equal to 3 days, or less than or equal to 2 days. Combinations of the foregoing ranges are possible (e.g., greater than or equal to 24 hours and less than or equal to 30 days). Other ranges are also possible. Note, however, in other embodiments, the release of the immunoadjuvant from the injectable composition may be relatively quick, e.g., as a burst release as described elsewhere herein.

Additionally, in some embodiments, the composition of the thermoresponsive, injectable gel, the presence and concentration of the immunoadjuvant, the presence and concentration of a radio-opaque label, and the resulting release profile (e.g., of the immunoadjuvant) are interconnected, making the parameter space and related design of experiments unexpectedly complex. Advantageously, the compositions described herein were formulated to provide injectable compositions having suitable retention properties of the gel, concentration of the immunoadjuvant, and concentration of the radio-opaque label with certain release profiles, according to some embodiments.

Some aspects of the present disclosure are generally related to methods, for example, for treating tumors. The injectable compositions described herein may, in some cases, be administered to a subject, e.g., such that the hydrophobic immunoadjuvant is delivered to the subject. For example, in some cases, the composition may be administered to the subject and a hydrophobic immunoadjuvant is released from the composition at a location internal to the subject. In accordance with some embodiments, administering the injectable compositions comprises injecting the compositions. Administration of the compositions and release of hydrophobic immunoadjuvants are described in more detail herein.

In some embodiments, the method comprises treating a tumor by injecting a composition intratumorally (i.e., into a tumor). In some embodiments, the method comprises treating a tumor by injecting a composition adjacent to a tumor. According to some embodiments, the composition may be injected into or near a tumor within a subject, e.g., in intra-abdominal, intrathoracic, intracranial or other anatomic compartments. In some embodiments, the composition may be a liquid at room temperature and a gel at 37° C. and may comprise a micellar structure comprising poly(lactide-co-glycolide-b-ethylene glycol-b-lactide-co-glycolide) (PLGA-PEG-PLGA) having a lactic acid (LA) to glycolic acid (GA) ratio of greater than or equal to 1:1, an immuno-adjuvant encapsulated within the micellar structure and present within the injectable composition at a concentration of greater than or equal to 1 mg/mL. According to some embodiments, the injecting may occur concurrently to another treatment of at least a portion of a tumor, for example, percutaneous cryoablation, microwave ablation, and/or irreversible electroporation of the tumor. In some embodiments, the efficacy of injection of the injectable composition (e.g., a localized immunostimulation) does not change in combination with second treatment, as compared to the instance without the second treatment.

The method may further include determining the position of the composition during injection using CT, in some embodiments. In some such embodiments, the composition may further include a radio-opaque label, e.g., encapsulated within the micellar structure, to facilitate the monitoring and/or determining of the position of the composition during injection.

As described above, in some embodiments, the composition may be a gel at temperatures above room temperature and less than or equal to 37° C. (or at other temperatures as described elsewhere herein). Accordingly, following injecting the composition, the composition may heat (e.g., due to the temperature of the body of the subject) and gel. In some such embodiments, the method comprises heating and gelling the composition at the injection location. Following, the gel may be retained at the injection location. According to some such embodiments, the method comprises retaining the gelled composition at the injection location for greater than or equal to 24 hours, greater than or equal to 2 days, and so forth as described elsewhere herein. Advantageously, in some such embodiments, directed injection of the composition may facilitate localized delivery and treatment of a tumor by an immunoadjuvant of the composition. In some embodiments, the localized delivery facilitates a local immunostimulation with a systemic effect (e.g., an abscopal effect).

For example, in some embodiments, the method comprises releasing the immunoadjuvant over a time of greater than or equal to 24 hours under physiological conditions. In some embodiments, the release of the immunoadjuvant is localized to an injection location, e.g., if the composition gelled and was retained at the injection location. The release, in some embodiments, may be logarithmic in nature.

Still in some embodiments, the method may further comprise degrading the composition. Degradation of the composition may occur naturally internal to the subject, for example, if the composition is biodegradable. An example degradation pathway includes hydrolysis of a polymer of the composition.

The following examples are intended to illustrate certain embodiments of the present invention, but do not exemplify the full scope of the invention.

### Example 1

In this example, a PLGA-PEG-PLGA copolymer for the controlled release of an example immunoadjuvant (e.g., imiquimod) in the presence of a radio-opaque label is described.

Percutaneous cryoablation is a common clinical therapy for metastatic and primary cancer. There are rare clinical reports of cryoablation inducing regression of distant metastases, known as the abscopal effect. Intratumoral immunoadjuvants may be able to augment the abscopal rate of cryoablation, but existing intratumoral therapies suffer from the need for frequent injections and inability to confirm target delivery, leading to poor clinical trial outcomes. To address these shortcomings, an injectable thermoresponsive gel-based controlled release formulation was developed for the FDA-approved Toll-like-receptor 7 (TLR7) agonist imiquimod (“Imigel”) that forms a tumor-resident depot upon injection and contains a contrast agent for visualization under computed tomography (CT). The poly-lactic-co-glycolic acid-polyethylene glycol-poly-lactic-co-glycolic acid (PLGA-PEG-PLGA)-based amphiphilic copolymer gel's underlying micellar nature facilitates high drug concentration and a logarithmic release profile that is additive with the neo-antigen release from cryoablation, facilitating the use of only a single injection. Rheological testing demonstrated the thermoresponsive increase in viscosity at body temperature and radio-opacity via microCT. Its ability to significantly augment the abscopal rate of cryoablation is demonstrated in otherwise immunotherapy resistant metastatic tumors in two aggressive colorectal and breast cancer dual tumor models with an all or nothing response, responders generally demonstrating complete regression of bilateral tumors in 90-day survival studies.

### 1 Introduction

In cancer treatment, the holy grail of locoregional therapies remains the promise of an abscopal effect. Abscopal refers to the phenomenon that a local therapy could induce therapeutic effect distantly in metastases, presumably secondary to immune activation. In interventional oncology, the abscopal effect has rarely been seen among patients referred for percutaneous cryoablation, a technique where a probe is placed under CT or ultrasound guidance and is used to freeze-thaw tumors. The abscopal effect seen here has been theorized from research models to be secondary to the neo-antigen burst release, and multiple pre-clinical studies have shown increased immunostimulatory effects particularly for immunotherapy sensitive patients. For example, at Massachusetts General Hospital, 18 patients identified with metastatic disease treated with immunotherapy within 90 days before or up to 30 days after percutaneous cryoablation. Of those, one patient with metastatic melanoma demonstrated a particularly sustained abscopal effect following percutaneous cryoablation of a pulmonary metastasis (FIG. 1C) manifested by regression of metastases in the contralateral lung and retroperitoneal lymph nodes. The combination with immunotherapy is actively being studied in clinical trials. For cryoablation, there is a significant need to find a method to make this rare but exciting abscopal effect more reproducible.

One potential method is to inject intratumoral immunoadjuvants into a single site of disease peri-cryoablation to activate the innate immune system to sample the neo-antigen burst. The innate immune system in turn activates and educates the adaptive immune system, namely, the subset of cytotoxic CD8+ T cells that are primarily responsible for immune-mediated tumor killing, ideally attacking tumor throughout the body. Following tumor recognition, CD8+ antitumoral activity is in turn maintained by systemic checkpoint inhibitor therapy. The inventors hypothesized that combining intratumoral immunoadjuvants with cryoablation may improve the reproducibility of the previously-rarely clinically seen abscopal effect. Most immunoadjuvants are small molecules, with rapid diffusion away from the tumor region. Although several immunoadjuvants for intra-tumoral injection are currently in clinical trials, including STING and TLR7/8/9 agonists, only a few have arrived in clinic. Among small molecules, only imiquimod is a clinically approved TLR7 agonist that increases dendritic cell activation and is clinically approved as a topical cream for treatment of basal cell carcinoma. It was reasoned that this drug could act as a proof-of-concept drug with high translational potential as many intratumoral drugs in development are similarly hydrophobic small molecules. In preclinical studies, imiquimod in combination with immunotherapy has shown some efficacy albeit requiring daily 50 μL injections with 1 mg mL⁻¹ imiquimod across 6 days showed improved survival. It was hypothesized that a logarithmic release of imiquimod over a similar number of days, that is, a larger release time around the ablation, followed by a logarithmic release, may improve the abscopal rate of cryoablation. The mechanism of action was expected to be due to dendritic cell recruitment from TLR 7 activation followed by T-cell activation and cell mediated killing (FIGS. 1D-1E).

Further, a significant hurdle to clinical translation remains the need for multiple repeated injections and lack of imaging confirmation. The injection of visceral organs or deep lymph nodes by interventional radiologists generally requires CT- or ultrasound guidance and procedural sedation. Without some imaging agent embedded, it is challenging to verify the targeted delivery to delivery or assess for off-target delivery during or after the procedure-which is critical for the evaluation of therapeutic efficacy and therapeutic window. To address these challenges an injectable, thermoresponsive, locally resident and imagable viscous gel to contain and deliver a high concentration of imiquimod, optimized for concurrent delivery with cryoablation, was developed. In two different syngeneic murine dual tumor models of checkpoint inhibitor resistant cancer, it was tested whether intratumoral Imigel injection rate significantly improves the abscopal rate of cryoablation.

### 2 Results

### 2.1 Development of a Gel Depot for Hydrophobic Immunoadjuvant Imiquimod (Imigel)

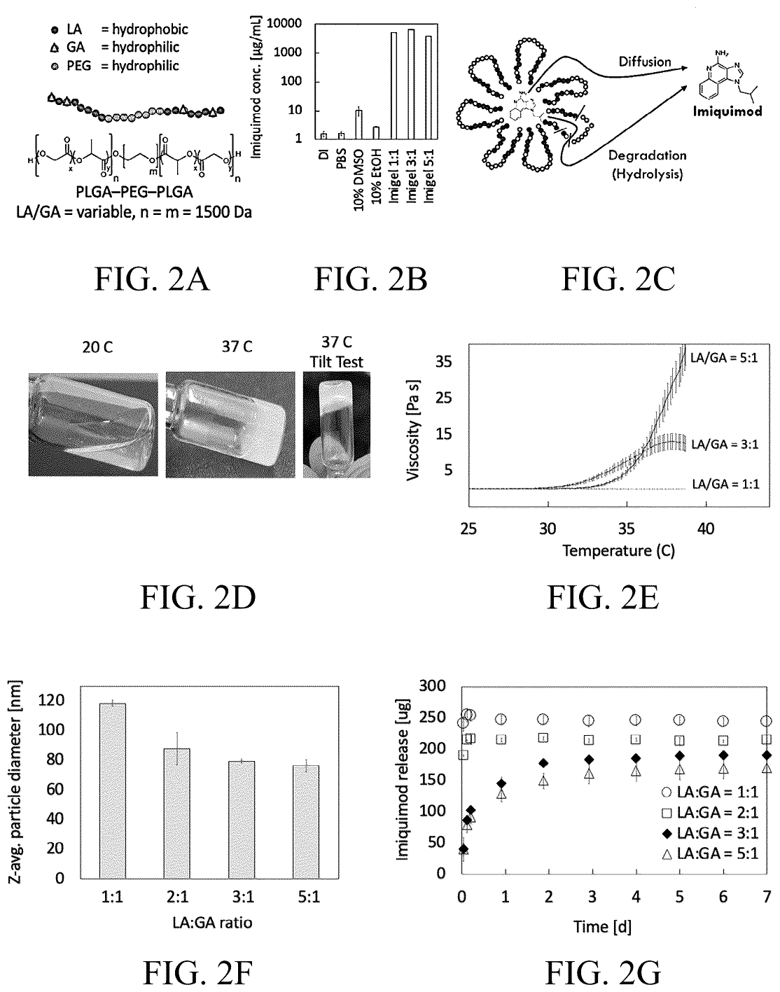

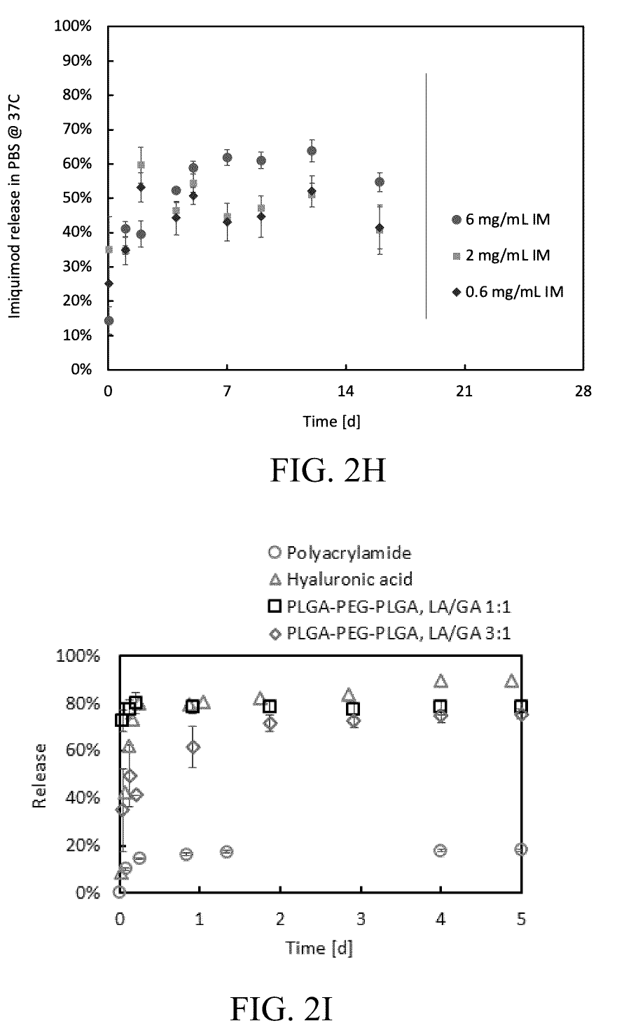

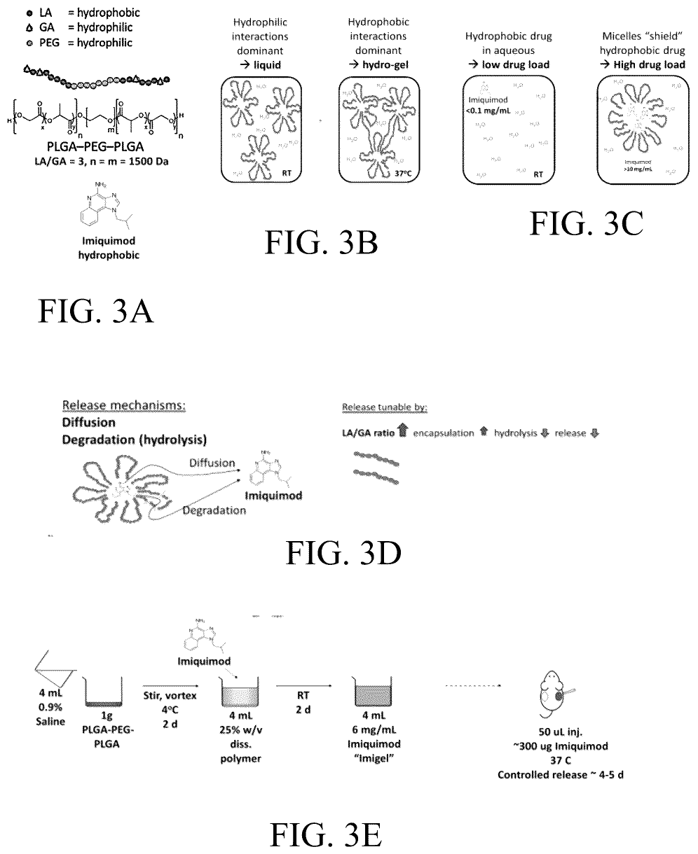

Multiple polymeric systems were investigated for the short-term controlled release of imiquimod. Given the constraints of injecting during an interventional oncology procedure, Thermoresponsive polymer with biocompatibility, injectability, and the ability to carry a high dose of drug in a relatively small volume were considered. To increase the translatability of the formulation, immunoadjuvants with proven clinical relevance, particularly imiquimod, and approved polymers with benign safety profiles were the focus. PLGA-PEG-PLGA is an amphiphilic triblock-copolymer composed of relatively hydrophobic (PLGA) and hydrophilic elements (PEG). The triblock structure facilitates two advantageous features of the controlled release formulation: first, it self-assembles into micellar nanoparticles around hydrophobic drugs, thereby dramatically increasing their aqueous solubility, and second, around body temperature this micellar structure creates a viscous gel (FIG. 2A, FIG. 3A). Both the gel formation and the drug encapsulation are largely guided by the PLGA copolymer structure, where the ratio of the lactic and glycolic acid monomers guides the hydrophobicity of the PLGA blocks. Lactic acid (LA) has a methyl side group, rendering it more hydrophobic than glycolic acid (GA). Capitalizing on this molecular handle to tune the polymer's amphiphilic properties, PLGA-PEG-PLGA polymers with varying LA:GA ratios were explored to tune hydrophobicity, and thus gel behavior, drug encapsulation, and drug release, with the goal of a multiday release to sustain a therapeutic effect.

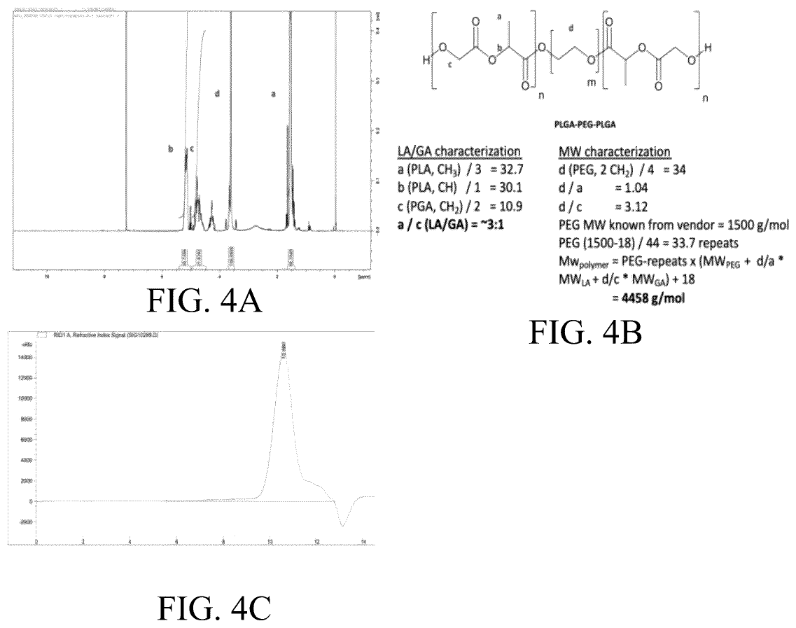

Imiquimod has a relatively low solubility in saline (e.g., around 0.002 mg mL⁻¹) and reported 1 mg mL⁻¹ solutions only exist as suspensions. These concentrations are well below a desirable concentration of 6 mg mL⁻¹ that is known to demonstrate improved survival. The use of PLGA1.5k-PEG1.5k-PLGA1.5k micelles was able to uniformly solubilize imiquimod at around 6 mg mL⁻¹ and thus in much larger concentrations than water, phosphate buffered saline (PBS), 10% DMSO/water or 10% Ethanol/water (FIG. 2B). Molecular weight and LA:GA ratios were confirmed by gel permeation chromatography (GPC) and by nuclear magnetic resonance (NMR) spectroscopy (FIG. 4). Increasing the hydrophobicity of the PLGA block by tuning the LA:GA ratio influences gelling behavior, where LA:GA=1:1 does not exhibit significant gelling due to insufficient hydrophobicity, whereas 3:1 and 5:1 show gelling points conveniently around physiological temperatures, as evidenced visually (FIG. 2D) and by rheometry (FIG. 2E). With increasing hydrophobicity (e.g., higher LA:GA ratio), particle size at room temperature decreases (FIG. 2F). Lower Critical Solution Temperature (LCST) marks the phase transition of thermosensitive polymers, which is ≈33.1° C. for LA:GA=3:1, while 1:1 did not exhibit an LCST and remained liquid up to 40° C.

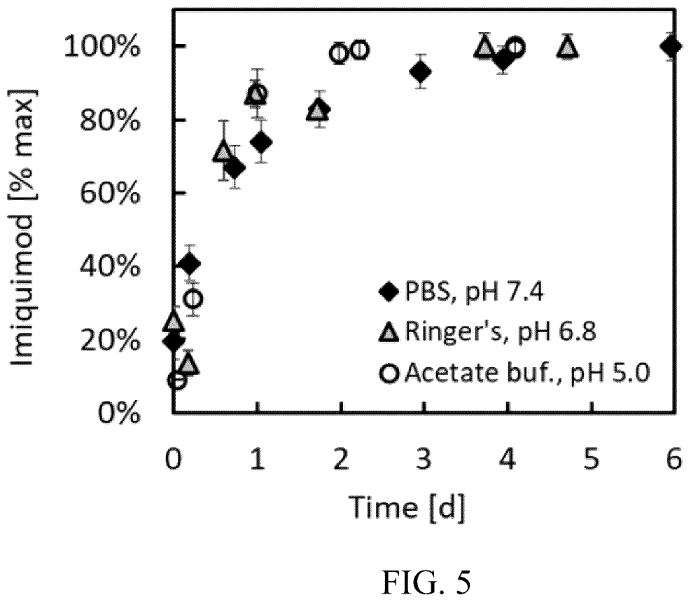

Drug release from PLGA-PEG-PLGA occurs primarily via diffusion until the polymer undergoes hydrolysis and thereby releases drug. The rate of release of the drug generally depends on polymer hydrophobicity, for example tuned by the ratio of LA:GA within the gel's PLGA block. Similarly, it was found that changing the LA:GA ratio also modulates the drug release kinetics of imiquimod, where LA:GA=1:1 leads to an almost instantaneous (burst) release of all drug while the more hydrophobic variants (e.g., where the LA:GA ratio is greater than 1:1) retain imiquimod for longer. The ratio can be used to obtain a multi-day release (FIG. 2F). The differences in plateau release amount may be a reflection of difference in imiquimod solubilization efficiency: all tested imigel formulations were prepared with 25% w/v PLGA-PEG-PLGA (1 gram polymer in 4 mL saline) and 6 mg mL⁻¹ imiquimod (24 mg added to the batch), however, the more hydrophobic formulations seem to uptake slightly less imiquimod, which would be congruent with the smaller micelle size observed. An increased hydrophobic core packing density can result in smaller micelles and thus lower drug load. Comparing the different formulations used herein, LA:GA=3:1 was chosen as the optimal tradeoff between gelation (1:1 and 2:1 did not gel in these examples) and drug load (5:1 showed less encapsulation). Relative release rate of imiquimod from LA:GA 3:1 imigel shows minimal changes with acidic pH (5 or 6.8) (FIG. 5). Although polymers can be designed for a linear release, the compositions described in this example were selected for a desired release profile, hypothesizing that having a relative majority of the drug delivered early (e.g., in the logarithmic release profile) would aid sampling of neo-antigens released during ablation. Note that the concentration of the immunoadjuvant present in the injectable composition did not affect the release rate into phosphate buffer solution (PBS) (FIG. 2H).

By comparison, hyaluronic acid and polyacrylamide showed almost immediate release (“burst”) or incomplete release (FIG. 2I).

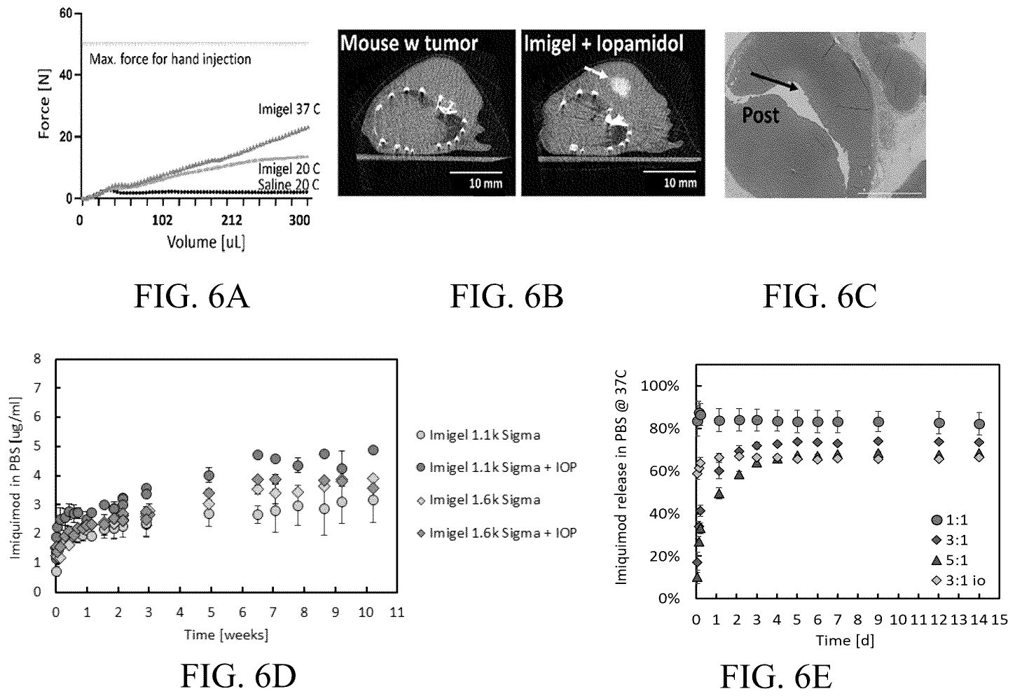

A desired feature for application of this technology in the clinic is the ability to be injectable through at least a 20-gauge needle, considering 18- to 22-gauge needles are common in clinical use. FIG. 6A demonstrates the injectability through a 22-gauge 15 cm Chiba needle of the room temperature (20° C.) version of the gel, whereas injectability generally requires higher force at 37° C. Force measurements demonstrate that either scenario generally requires forces well below the maximum 50 N suggested for an average healthcare worker's hand injection.

To facilitate delivery confirmation and for the clinician to be able to assess technical success of the procedure, the feasibility of incorporation of a radio-opaque contrast agent into the gel was explored. Imigel incorporated 100 mg mL⁻¹ of hydrophilic Iopamidol, an iodinated contrast agent, to facilitate the visualization of the injection in mice under micro-CT immediately after injection (FIG. 6B). The concentration of iodine for visualization depends on CT scanner clinically, but may be as low as 2 mg mL⁻¹, which can likely be selected based on the use case and organ. Immediate post-injection histologic evaluation of resected tumor post-injection demonstrates how the injected gel forms a depot within the tumor, creating a cleft in the tumor tissue (FIG. 6C). Additionally, the presence of the iopamidol may change the release kinetics of the immunoadjuvant from the injectable hydrogel, as evidenced by the release profiles of imiquimod from the injectable compositions with and without iopamidol in FIGS. 6D-E.

### 2.2 Imigel Significantly Improves the Abscopal Efficacy of Cryoablation in Both Colorectal and Breast Cancer Immunotherapy Resistant Mouse Models

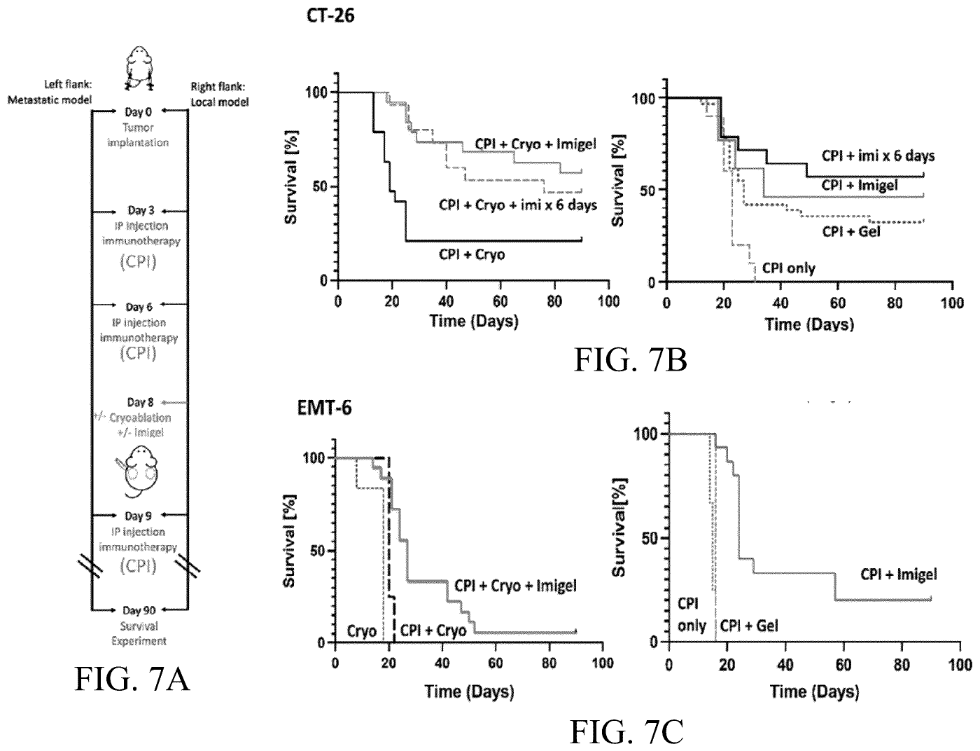

Syngeneic dual flank implanted tumor models for assessment of Imigel's performance in increasing the response rate to traditional checkpoint inhibitor (CPI) therapy in CPI-resistant tumor models were used, where CPI alone leads to zero response (i.e., no survival), and compare the results also to a combination with cryoablation and the 6× daily injection scenario. CT-26 tumors are a micro-satellite stable murine-derived colon cancer line that is known to be moderately responsive to CPI therapy. EMT-6 is a murine breast cancer that is poorly responsive to CPI. In these dual tumor models, intra-tumoral therapy is applied to only one of the two implanted tumors (i.e., the ipsilateral tumor), while response is monitored in both the intervened and non-intervened (contralateral) tumor (FIGS. 7A-7C). In all cases shown in this experiment, the two tumors implanted in each mouse were of the same cell line to create a consistent and readily assessed model of distant disease. In general, all treatments were done in the background of systemic checkpoint inhibitor therapy.

In both the CT-26 and EMT-6 model, a significant increase in 90-day survival and abscopal rate of mice when cryoablation is combined with imigel co-injection was seen when compared to cryoablation and CPI. For the CT-26 model, cryoablation+imigel+CPI had a 90-day survival percentage of 57% (10/19) compared to CPI alone, 0% (0/10) (p<0.0001 Log rank test), or CPI+cryoablation of 21% (4/19) (p=0.0003 Log rank test).

For the EMT-6 model, at 90 days cryoablation+imigel+CPI demonstrated a higher 90-day survival (5.6%, 1/18) compared to CPI alone (0%, 0/7) or CPI+cryoablation (0%, 0/8), (p=0.0003 Log rank test). (FIG. 7C).

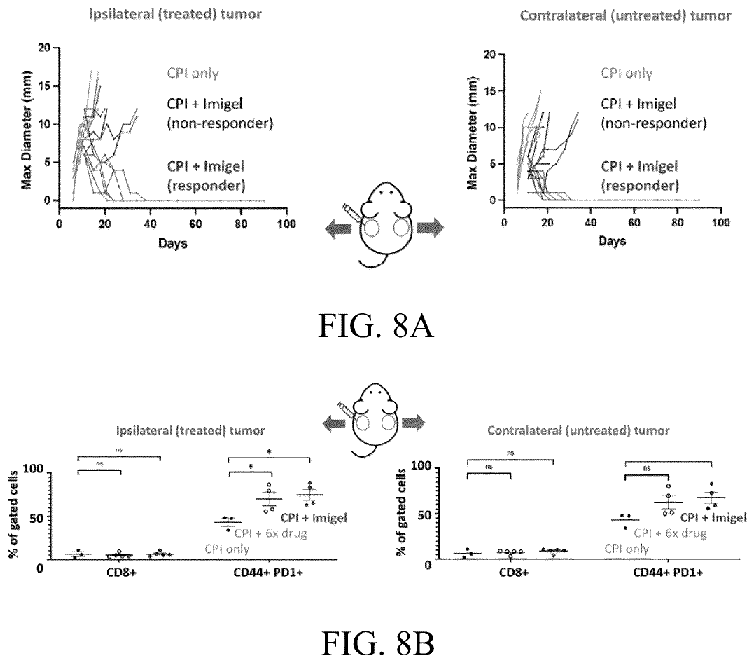

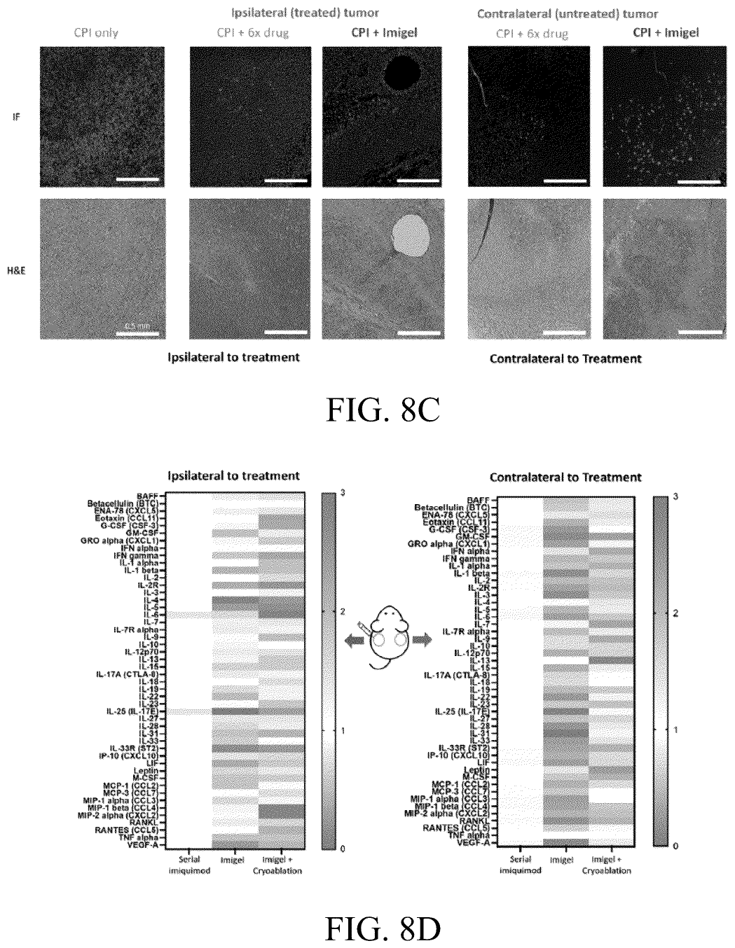

Intriguingly, in each model a significant effect was also seen from the imigel itself plus checkpoint inhibition without cryoablation. At 90 days, in the CT-26 model, the addition of Imigel to checkpoint inhibition immunotherapy significantly increases 90-day survival to 46% (6/13) compared to the control experiments CPI alone 0% (0/10) (p=. 0045 Log rank test). Similarly, addition of the gel (without imiquimod)+CPI has a significant effect with a 90 day 35% (11/31) compared to CPI alone, (p=. 02, Log rank test). In the CT-26 model, the imigel+CPI versus gel+CPI are not significantly different (46% vs 35% 90-day survival p=. 54, Log rank test). Adding cryoablation to imigel however did show a significant increase in survival compared to CPI+gel alone (57% vs 35% p=0.04 Log rank test), suggesting some degree of an additive effect of the triple combination. As expected, Imigel acts similarly to serial imiquimod injection. For example, the efficacy with imigel is similar to the efficacies with 6 daily injections of imiquimod alone. (CPI with cryoablation and serial imiquimod, 46.7% (7/15) 90-day survival) and (serial imiquimod with CPI, 57% (8/14) 90-day survival) (FIG. 7B). Again, all mice that survived to 90 days had full tumor regression, while CPI only and treatment non-responders had significant tumor growth until euthanasia (FIG. 8A).

The interesting effect of the gel alone seen in the CT-26 model does not replicate in EMT-6, with CPI+gel having a 0% survival effect (0/12), at 90 days. Similarly, in the EMT-6 model there was limited 90-day survival when using CPI alone (0%, 0/7) or CPI+cryoablation (0%, 0/8). Instead, the breast cancer model appears to generally require imiquimod for efficacy of inducing an abscopal effect. Imigel significantly improved response to 20% (3/15), and triple combination therapy (immunotherapy+cryoablation+Imigel) had a 5.6% (1/18) 90-day survival compared to gel (p<0.0001 Log Rank Test), CPI alone (p<. 0001 Log rank test), or CPI+cryoablation (p=. 0003 Log rank test) (FIG. 7C). Imigel+CPI versus triple combination therapy was not significantly different. For toxicity, no mice demonstrated significant weight loss or signs of systemic sepsis like findings.

### 2.3 CT-26 Model Intratumoral Flow Cytometry and Cytokine Release Analysis

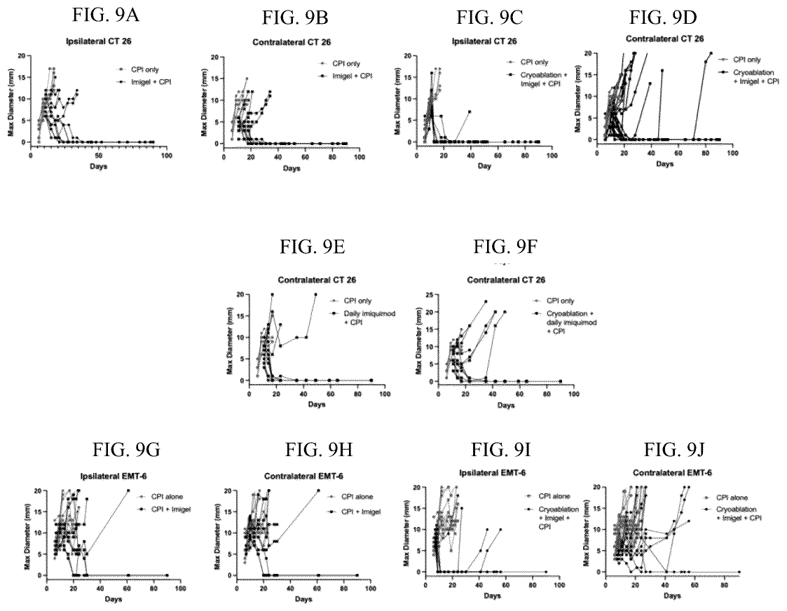

When evaluating tumor growth curves, interestingly, in both models, this increase in survival is due to an all-or-nothing abscopal response, with either complete regression in the bilateral tumors (treated and untreated) or almost no effect for imigel+CPI (FIG. 8A). Almost all of the imiquimod exposed treatment groups demonstrated this complete regression effect for the majority of treated mice if they responded. A few mice in the CT-26 or EMT-6 model for cryoablation+imigel or cryoablation+serial imiquimod demonstrated later onset recurrence in the contralateral tumors (FIG. 9).

Given these results, the immune response profile of immune stimulation from CT-26 colorectal cancer models from imigel application and systemic checkpoint inhibitor therapy was explored. Evaluation of the ipsilateral injected and contralateral non-injected tumors by flow cytometry demonstrated no significant increase in the absolute number of CD8+ T cells relative to CPI-only treated mice. However, a significant increase in the percentage of activated CD8+ T cells was seen, among both imiquimod and Imigel treated mice as assessed by the percentage of dual CD44+PD1+ T-cells among CD8+T-cells. A significant increase in T cell activation was seen in both injected and non-injected tumors with Imigel (p=0.01 ipsilateral and p=0.03 contralateral) compared to CPI only (FIG. 8B). Granzyme B can be used as a marker of CD8 T activation. Immunofluorescence of this marker qualitatively, but non-quantitatively, demonstrates increase in cytotoxic cell activation within bilateral tumors with imigel treatment (FIG. 8C). Quantifying this visual result is limited by the potential sampling bias from slice artifact.

In evaluating cytokines released by the treatment, of note, imiquimod itself is known to induce a significant increase in IL-1, IL-1RA, IL-6, IL-8, IL-10, IL-12p40, TNF, IFN-alpha, G-CSF, among others. As such, the effect of the gel and cryoablation compared to serial imiquimod ipsilateral fold change release compared to ipsilateral serial imiquimod was evaluated. Compared to imiquimod alone, imigel notably increased TNF-alpha (1.9×) (pro-inflammatory), IL-4 (3.9×), and IL-5 (2.5×) (an eosinophilic response, respectively), while adding cryoablation demonstrated an increase in pro-inflammatory cytokines TNF-alpha (1.9×), IL-6 (11.8×), and CXCL-2 (3×), also eosinophilic IL-5 (2.6×), IL-4 (2.5×), and pro-macrophage inflammatory markers CCL-4 (3×) and IL-25 (2.4×) (Figure SD)). Comparatively in the contralateral cytokine evaluation, a general mild decrease in cytokines in imigel and with cryoablation compared to serial imiquimod was seen. This may be due to the differing release profile of Imigel versus daily imiquimod. The cytokine release suggests that the gel and cryoablation are complementary with the improved efficacy from the robust IL-6 response seen in cryoablation.

### 3 Discussion

Cryoablation as a locoregional therapy has offered the tantalizing promise of a local therapy inducing distant effects on metastatic disease. This effect, though, has been rare clinically, particularly among checkpoint inhibitor resistant cancers, both pre-clinically and clinically. Co-delivered imigel provides a potential method to overcome local barriers to the anti-tumoral immune response, facilitate systemic anti-tumoral immunity and increase the abscopal effect of percutaneous cryoablation+CPI for patients with metastatic disease.

While many intratumoral immunotherapy drugs and drug types are in development, their effective delivery remains underappreciated. Limitations to intratumoral immunotherapy have become clear, which include the leaking of drug away from target, the need for frequent injections, and the inability to confirm on-target delivery of therapy. These limitations were addressed by the compositions developed in this example and described elsewhere herein by the development of an injectable controlled-release immunoadjuvant depot with the capacity to facilitate image-guided delivery and confirmation, which thereby can unlock the full potential of existing immunoadjuvant drugs and those in development. The use of these imagable depot-forming hydrogel can be expanded to many intratumoral drugs in development, especially hydrophobic small molecules. In this study, drug delivery was performed around co-delivery with an ablative therapy, with the goal for a logarithmic release to coincide with antigen release.

Interestingly, the ability of imigel itself to induce an abscopal effect itself was a welcome surprise. By results of the survival study, the contribution of imiquimod versus cryoablation appears to be different based on the tumor type. In the CT-26 colorectal cancer model, the combination of cryoablation and imigel appears additive with at least some effect from the gel itself. In the EMT-6 model, the effect of imigel is nearly identical to that of cryoablation+imigel itself. These results are consistent with cryoablation and imigel being potentially additive and complimentary with imigel and the gel providing a significant boost.

The mechanism of TLR 7 agonists such as imiquimod are well known. Briefly, these innate markers activate dendritic cells and other antigen presenting cells within tumors. When these cells are activated within a tumor they travel to tumor-draining lymph nodes to in turn activate cytotoxic CD8 T cells. Cytotoxic T cells then are directed to tumor to mediate tumor-cell killing. The evaluation of the tumor infiltrate herein similarly matches these expectations of increases in activated CD-8 T-cells. Interestingly, the all-or-nothing abscopal response model provides a unique pre-clinical model to study what other stochastic factors are occurring to explain why some tumors completely regressed and others did not and is currently an active area of study.

The cytokine analysis described in this example suggests both a pro-inflammatory and interesting eosinophilic response from cryoablation and the gel itself over the usual effects of imiquimod, and it is interesting that the gel itself can induce that response, possibly explaining its ability to also reproduce an abscopal effect even without imiquimod in the CT-26 model. The eosinophilic effect could potentially be explained with the immunogenicity of certain polymers including PEGs. In comparison, other groups have focused on augmenting cryoablation via trapping antigens nearby with nanoparticles to augment the abscopal effect, or shaping the ice-ball with nanomaterial adjuncts, a complementary approach. Given the gel appears to be mimicking the eosinophilic response seen from cryoablation, this example study provides the basis for percutaneous gel depots to potentially get the same effect from cryoablation itself without the second ablation therapy.

A notable challenge with working with imiquimod is its poor aqueous solubility, which limits large concentrations in saline to be injected as suspensions with impractical heterogeneity. Prior attempts at intratumoral imiquimod delivery using PLGA microparticles have been limited to doses of around 0.1-0.4 mg mL⁻¹ and generally required combination with photoablation for efficacy, thereby limiting use for the much more commonly seen deeper lesions. Other attempts have been made to use higher activity but non-clinical TLR7/8 agonists such as resiquimod (R848) or MEDI9197 to get around these solubility limitations, however, even using polymers such as Pluronics (PEO-PPO-PEO) or PLGA nanoparticles, multiple injections were generally required, for example ten injections with drug loadings ranging from 20-75 ug of resiquimod or novel variants thereof per injection. A large depot of imiquimod was delivered through the encapsulating, micellar structure of PLGA-PEG-PLGA (effectively increasing the aqueous solubility of imiquimod by approximately 2000-fold).

While polymers comprising PLGA and PEG have been used previously for chemotherapy delivery and has been well tolerated clinically, the formulation has not been shown to have significant and/or clinical efficacy. Moreover, previous evaluations of PLGA-PEG-PLGA thermo-gels for chemotherapy formulations encountered challenges with achieving full-tumor coverage. However, the inventors of the injectable composition described herein, in the context of concurrent treatment with checkpoint inhibitors, discovered that complete tumor coverage is not necessary, as it is the subsequent activation of cytotoxic CD8 T cells that mediates tumor killing, locally, and distally. The inventors discovered that by combining a logarithmic release of imiquimod with an ablative therapy, a single dose therapy may be able to be found.

The radio-opaque intratumoral imigel advantageously facilitates clinical translation for deeper lesions. Imigel was shown to be able to concurrently incorporate an iodinated contrast agent, Iopamidol, and demonstrate optimal injection into the tumor ideally avoiding issues of mis-targeting as reported clinically. This visualization opens the door for intratumoral immunotherapy to deep visceral tumors where mis-targeting can be significantly more problematic.

Limitations for the study include utilization of a murine tumor model with tumor implantation via xenograft. By selecting and treating tumors that had measured to 6 mm while immunotherapy was already active, tumors chosen were immunotherapy resistant to begin with, suggesting that the results likely under-estimate the efficacy of combination therapy in immunotherapy naïve tumors. In addition, as compared to more novel immunoadjuvants, an FDA-approved drug imiquimod was utilized, that has a broader activation profile than an even more targeted agent might have. This limitation reduced the biochemical control in situ but provides for likely greater translatability to patients.

Overall, a single intratumoral injection of the engineered logarithmic-release Imigel in combination significantly increases the abscopal rate of cryoablation in two different immunotherapy resistant tumor models. As suggested by the immune-assays, Imigel achieved these improved outcomes through increased activation of cytotoxic CD8 T cells, and in the ipsilateral tumor, an increased TNF-alpha, IL-4, and IL-5 release. This strategy brings personalized therapy to cancer patients with metastatic disease, effectively immunizing them to their own cancer via a local therapy that could be done concurrently with standard of care percutaneous cryoablation.

### 4 Experimental Section

### Statistical Analysis

In murine survival studies, statistical analysis was done using Kaplan-Meier and Log-rank tests. Any comparison of means was done using a 2-tailed t-Test. Statistical studies were done in Prism (Graphpad, Boston, MA)

### Ethics

All studies were completed under an approved animal care and use protocol, 2017N000163, as well as an IRB protocol, 2020P003099, for the retrospective chart review.

### Materials

PLGA (1500 g mol-1)-PEG (1500 g mol-1)-PLGA (1500 g mol-1) copolymers with lactide-to-glycolide ratios (LA/GA)=1:1, 2:1, 3:1, 5:1 were purchased from NanoSoft Polymers (Winston-Salem, NC) and stored at 4° C. NMR and GPC quality control data provided by NanoSoft Polymers. The copolymer, imiquimod (98%, Sigma-Aldrich, St. Louis, MO), Iopamidol (99.8%, MedChemExpress (Monmouth Junction, NJ), and Dulbecco's Phosphate Buffered Saline (DPBS, FisherScientific, Hampton, NH) were used as received.

### Imigel Preparation

Typically, 1 g of PLGA-PEG-PLGA was dissolved in 4 ml 0.9% saline solution (Baxter, Deerfield, IL) to obtain a 25% weight/volume (w/v) concentration at 4° C. over 2 days and vortexed/stirred daily. Importantly, the normal saline cannot contain the preservative phenol, commonly found in preservative containing commercial saline solutions. After a homogeneous hydrogel solution was observed by stirring with a spatula, imiquimod was added at 24 mg to the batch, the dispersion was vortexed and allowed to dissolve at room temperature over 2 days. Some of the gels received Iopamidol as contrast agent at 100 mg mL⁻¹, which was left to disperse or dissolve over 1 day. The resulting Imigel or Imigel+Iopamidol was stored at room temperature, shielded from evaporation, and used within 1 week from full dissolution to prevent hydrolysis. (FIGS. 3A-3E).

### In Vitro Release Studies

Drug release kinetics of imiquimod from various imigel formulations were studied by placing aliquots of drug-loaded hydrogel into 50 mL conical tubes at 100 rpm at 37° C., releasing drug into DPBS. 50 μL of Imigel were added to the bottom of 50 mL conical Falcon tubes or in a dialysis tubing (D-tube Dialyzer Maxi, MWCO 3.5 kDa) at room temperature and brought to gel state by incubating at 37° C. for 30 min while DPBS as receiving fluid was preheated at the same temperature. After Imigel solidified, 50 mL DPBS (pH 7.4), Lactated Ringers (pH 6.8, Vetivex), or Acetate Buffer (pH 5, 0.03 m Acetic acid, and 0.07 m Sodium Acetate in DI water) were added slowly to the conical tubes. These containers were then incubated at 37° C. under shaking at 100 rpm. 400 μL samples were taken from the receiving fluid with a 1 mL syringe through 0.2 μm filters (PALL super membrane low protein binding non-pyrogenic) to avoid accidental removal of gel. Concentration of imiquimod in the samples was assessed eluting the samples on an Agilent 1100 high-performance liquid chromatography (HPLC) with UV detector at 242 nm over an Eclipse XD8-C18 column (150×4.6 mm, 3.5 μm pore size) with an acetonitrile/water gradient from 5/95 to 95/5 over 6 min at 1 mL mi-1 n. Formic acid 0.1% (v/v) was added as stabilizer to both the organic and aqueous phases, using millipore water as the aqueous phase and ACN as the organic phase. The injection volume was kept constant at 5 μL. Concentrations were calculated from UV signals using a calibration curve of known concentration imiquimod standards in DMSO/Acetonitrile.

### Rheometry

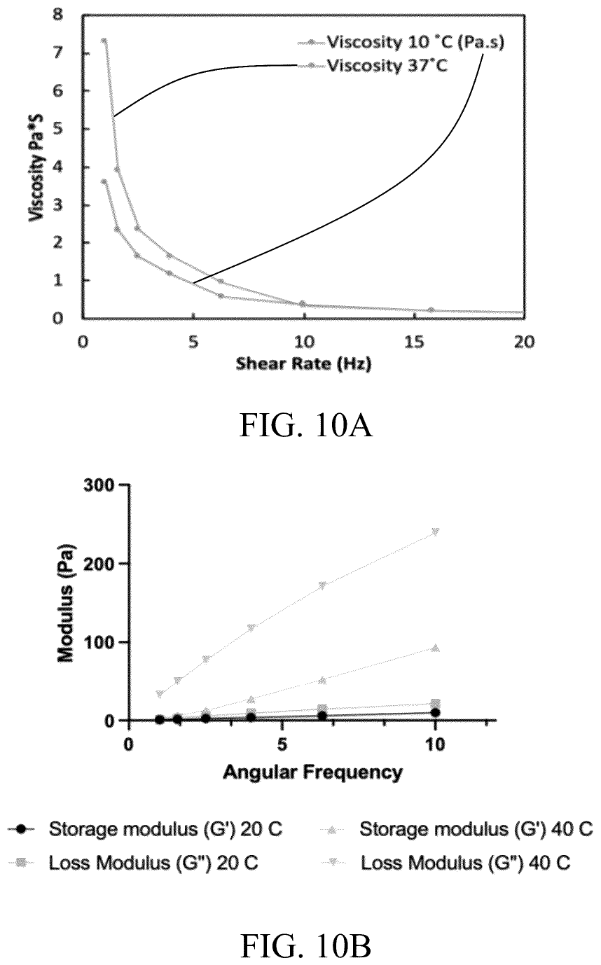

Gel rheology was characterized using a Discovery Hybrid Rheometer (TA instruments, New Castle, Delaware) using 20 mm parallel plates modified with 600 grit adhesive-backed sandpaper to eliminate slip effects. Frequency sweeps at 10 and 40° C. were performed to measure storage and loss modulus at a strain of 1%. Flow sweeps were performed at varying shear rates from 0.1-100 1/s at 20° and 37° to compare viscosity and shear stress. These same flow sweeps were done after injection through an 18-gauge needle for the extreme ratios of 1:1 and 5:1. (FIGS. 10A and 10B).

### Injection Pressure Measurements

300 μL of Imigel at room temperature (approximately 20° C.) and after 30 min at 37° C. incubation, as well as normal saline at room temperature were compressed in a 3 mL syringe at 200 μL s⁻¹ through a 22-gauge 15 cm Chiba needle using an Instron machine. A 22-gauge 15 cm Chiba needle was considered clinically to be the likely most difficult and clinically relevant injection scenario.

### Dynamic Light Scattering

Particle size distributions were analyzed on a Malvern Zetasizer. 500-1000 μL aliquots of imigel formulations were evaluated in 70 μL plastic cuvettes (BRAND, 70-850 μL) at room temperature and 37° C. Material parameters were used for PEG as the material and water as the solvent. Particle size averages and distributions were then taken and compared.

### Injection Rheometry and DLS Measurements

Rheometry and DLS data were collected pre and post injection through an 18-gauge needle. For the pre-injection measurement, approximately 800 μL of imigel (1:1, 5:1) were pipetted into a cuvette and measured in the DLS at 25° C. with the aforementioned settings. 500 μL of imigel were pulled up from the pre-injection cuvette using an 18-gauge needle and injected into a separate cuvette for DLS measurement with the aforementioned settings at 25° C. These samples were then measured in the rheometer using the aforementioned settings.

### MicroCT Scans

Mice (N=3) were injected subcutaneously dorsally with CT-26 cells (1-2 million cells) in Matrigel. After 14 days, the tumors were injected with 50 μL 6 mg mL⁻¹ imigel containing Iopamidol (100 mg mL⁻¹) and imaged with a microCT scanner to confirm the radio-opacity of the gel and visibility in vivo.

### Cell Culture

CT-26 and EMT-6 tumor cells lines (ATCC, Manassas, VA) were cultured for in vivo mouse models. These cells were cultured in flasks of T125 mL RPMI 1640 (Sigma Aldrich, St. Louis, MO) medium supplemented with 10% fetal bovine serum (FBS) (Sigma Aldrich, St. Louis, MO) at 37° C. in a 5% CO2 atmosphere.

### Dual Tumor Model In Vivo Experiments

Per experimental arm 10-15 Balb c⁻¹ mice were injected with approximately 10⁶ cells of CT-26 or EMT-6 cells, day 0 (FIG. 6) in bilateral flanks. Immunotherapy (anti-PD-1 (3.9 mg mL⁻¹), anti-CTLA-4 (2.2 mg mL⁻¹) co-immunotherapy was given intraperitoneally (Sigma-Aldrich) every 3 days from day 3-12. Mice with primary and contralateral tumors of at least 6 mm were chosen to be randomized to various treatment arms evaluating CPI with and without cryoablation, imiquimod drug-only injection, PLGA-PEG-PLGA only injection, or imigel (3:1) injection. The imigel, gel alone, or serial imiquimod suspensions were injected as 50 μL injections using a 26-gauge needle. For cryoablation, parameters included a 30 s freeze, followed by 2 min thaw, followed by a 30 s freeze with goal of approximately 50% tumoral freezing. When using cryoablation, the drug formulations were injected 5 min post final freeze, with a mild pause during injection to ensure no push back via the tract into the remaining viable tumor. Imiquimod alone was prepared as a suspension by 30 min of sonication at 1 mg mL⁻¹ in normal saline. Injections for imiquimod alone were done as a total 50 μL 1 mg mL⁻¹ injections once per day for six total doses. Specifically, Imigel delivered a total of 300 μg in one injection in two 25 μL injection depots within the tumor at the same injection session for only one session. Imiquimod suspensions were delivered as two 25 μL depots at 1 mg mL⁻¹ (50 μg injection⁻¹ day⁻¹) over the course of 6 days leading to a total 300 μg dose. Mice were then followed for tumor size, with tumor measurements twice weekly for up to 90 days when the mice were sacrificed. Mice were sacrificed if the tumor grew to be at least 2 cm in size, ulcerated, or the mice showed other signs of stress, such as >20% weight loss.

### Flow Cytometry and Cytokine Analysis

Additional groups of 4 mice were injected with CT-26 and treated as above. 6 days post local intervention, mice were euthanized, and ipsilateral and contralateral tumors excised. In all cases of this example, tumor samples were minced and then incubated in a collagenase solution (1 mg mL⁻¹ in RPMI—1640) for approximately 45 min at 37° C. Tumors were processed into a single cell suspension using a 70 μm cell strainer.

Supernatant from the above process was analyzed for cytokine and chemokine levels. Cytokine and chemokine levels were measured using a preconfigured ProcartaPlex Multiplex Immunoassay (ThermoFisher Scientific), according to the manufacturer's instructions. Briefly, magnetic beads were added to a 96-well plate and then washed with 1× Wash Buffer. Universal Assay Buffer was added to the wells, followed by the samples, antigen standards, and blanks (additional Universal Assay Buffer). The plate was incubated for 2 hours at room temperature on a plate shaker at 500 rpm. The wells were then washed with 1× Wash Buffer and detection antibody was added, followed by incubation for 30 min at room temperature on a plate shaker at 500 rpm. SAPE (streptavidin, R-phycoerithrin conjugate) was then added, followed by incubation for 30 min at room temperature on a plate shaker at 500 rpm. The plate was then washed, and Reading Buffer was added to each well. The beads were resuspended by shaking the plate at 500 rpm for 5 min at room temperature. The plate was then run on a Luminex 200 instrument (Luminex Corporation).

For flow cytometry, using the cells harvested as above, cell viability was assessed by trypan blue staining, and 1×10⁶ viable cells were stained for flow cytometry analysis. Cells were washed with PBS and stained with a 1:1000 solution of ZOMBIE Aqua (from ZOMBIE Violet Fixable Viability Kit, Biolegend) and incubated on ice for 15 min. After washing twice with cell staining buffer (Biolegend), the following extracellular antibodies were added: CD45.2-FITC (clone 30-F11, Biolegend), CD3-Pacific Blue (close 17A2, Biolegend), CD8a-Brilliant Violet 421 (clone 53-6.7, Biolegend), CD44-APC/Cy7 (clone IO7, Biolegend), PD-1-APC (clone 29F.1A12, Biolegend) at 100 μL total volume diluted with cell staining buffer and incubated for 20 min on ice in the dark. Cells were resuspended in cell staining buffer, and the samples were analyzed on an Aurora (Cytek Biotechnology). Data were gated using FlowJo software (FlowJo LLC). Doublets and cell clumps were excluded by gating along the 1:1 line for forward scatter height (FSC-H) versus forward scatter area (FSC-A). Cellular debris and dead cells were excluded by side scatter area (SSC-A) versus FSC-A and viability stain, and immune cells were then selected based on CD45 expression. Gating for individual markers was determined by fluorescent minus one (FMO) control panels and unstained controls. Gates were then confirmed using backgating.

### Immunofluorescence

Immunohistochemical staining of formalin-fixed paraffin-embedded excised tissues was performed. Antigen retrieval was conducted in citrate buffer (EMD Millipore, Burlington, MA) and blocking was conducted with Dako Protein Block Serum-Free buffer (Agilent, Santa Clara, CA). Cell nuclei were visualized with DAPI (1 μL/5 mL PBS) (BioLegend, San Diego, CA). Granzyme B expression was visualized with mouse monoclonal anti-granzyme B antibody (200 μg mL⁻¹, diluted 1:100 in PBS) (sc 8022) conjugated to Alexa Fluor 546 (Santa Cruz Biotechnology, Dallas, TX). Fluorescent images were captured on Biotek Cytation 5 Imaging Reader (Agilent, Santa Clara, CA) with constant LED exposure, integration, and camera gain for all tissues.

### Example 2

In this example, a PLGA-PEG-PLGA copolymer for the controlled release of another example immunoadjuvant (e.g., imiquimod) is described.

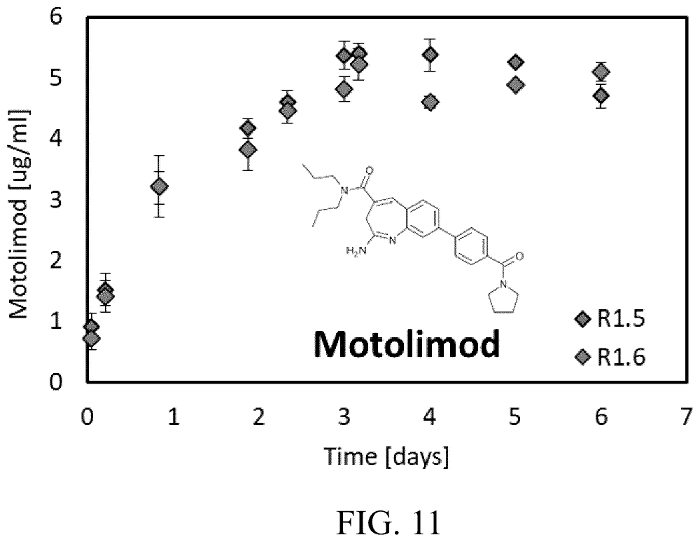

An injectable composition as described in EXAMPLE 1 was formulated, but in this example, the immunoadjuvant present was motolimod into PBS. FIG. 11 shows the release profile of motolimod from the PLGA-PEG-PLGA having blocks with molecular weights of 1600-1500-1600 and 1500-1500-1500. The release profiles are relatively extended, as in the case with imiquimod, facilitating an extended release of the immunoadjuvant within a subject, with motolimod having an elevated encapsulation around 6 mg/mL.

### Example 3

This example describes the efficacy of various radio-opaque labels.

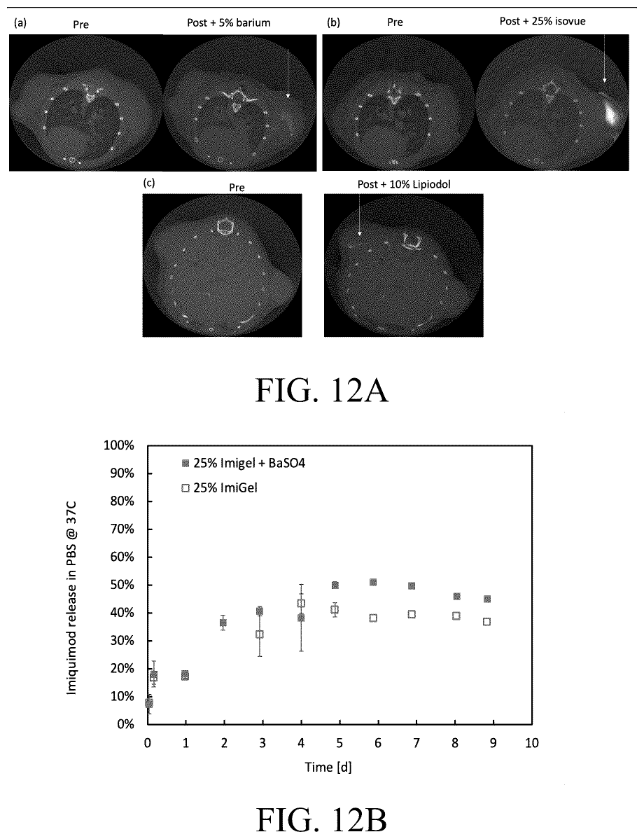

Injectable compositions as described in EXAMPLE 1 were formulated. The compositions included barium, iopamidol (Isovue), or lipiodol as radio-opaque labels. FIG. 12A shows CT scans taken of a subject before and after injecting the injectable compositions containing one of the radio-opaque labels. Barium and iopamidol work as expected, but lipiodol crashed out and fully settled. Additionally, FIG. 12B shows release a release profile of imiquimod from an injectable composition in the absence and presence of barium sulfate in the composition in phosphate buffer solution at 37 degrees C. This plot shows barium sulfate does not significantly change the release profile of imiquimod.

### Example 4

This example describes the behavior of injectable compositions having different blocks with different molecular weights.

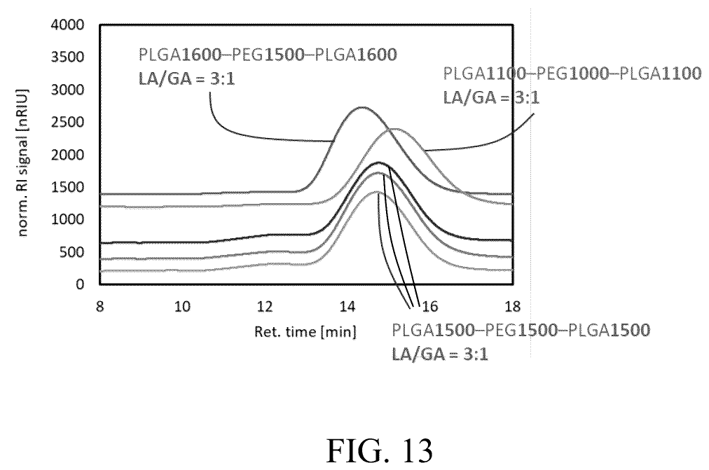

FIG. 13 shows the gel permeation chromatography of three different injectable compositions. Each composition comprises a block copolymer of PLGA-PEG-PLGA as described in more detail elsewhere herein. Table 1 describes the properties of each polymer at different temperatures.

TABLE 1
Properties of injectable compositions at various temperatures.

| Molecular weight | Molecular weight | At room | |
|---|---|---|---|
| of PLGA blocks | of PEG block | temperature | At 37° C. |
| 1600 | 1500 | Liquid | Solid |
| 1100 | 1000 | Viscous liquid | Solid |
| 1500 | 1500 | Liquid | Gel |

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. An injectable composition configured for intratumoral drug delivery, the injectable composition comprising:
a micellar structure comprising poly(lactide-co-glycolide-b-ethylene glycol-b-lactide-co-glycolide) (PLGA-PEG-PLGA) having a lactic acid (LA) to glycolic acid (GA) ratio of greater than or equal to 3:1;
a hydrophobic immunoadjuvant encapsulated within the micellar structure; and
a radio-opaque label present in the composition in an amount greater than or equal to 0 mg/mL and less than or equal to 200 mg/mL,
wherein the injectable composition is capable of encapsulating the hydrophobic immune-adjuvant at a concentration of greater than or equal to 1 mg per 1 mL of injectable composition,
wherein the molecular weight of each PLGA is greater than or equal to 500 and less than or equal to 2000,
wherein the molecular weight of the PEG is greater than or equal to 500 and less than or equal to 2000,
wherein the injectable composition is a liquid at room temperature and is a gel at 37° C., and
wherein the immune-adjuvant exhibits an extended release profile from the injectable composition over greater than or equal to 12 hours as measured in phosphate-buffered saline at 37° C.

2. An injectable composition configured for drug delivery, the injectable composition comprising:
a micellar structure comprising poly(lactide-co-glycolide-b-ethylene glycol-b-lactide-co-glycolide) (PLGA-PEG-PLGA) having a lactic acid (LA) to glycolic acid (GA) ratio of greater than or equal to 1:1;
a hydrophobic immuno-adjuvant encapsulated within the micellar structure; and
a radio-opaque label present in the composition in an amount greater than or equal to 0.01 mg/mL and less than or equal to 200 mg/mL,
wherein the molecular weight of each PLGA is greater than or equal to 500 and less than or equal to 2000, and
wherein the molecular weight of the PEG is greater than or equal to 500 and less than or equal to 2000.

3. An injectable composition as in claim 1, wherein the radio-opaque label is present in the composition in an amount greater than or equal to 0 mg/mL and less than or equal to 100 mg/mL.

4. An injectable composition as in claim 1, wherein the radio-opaque label is present in the composition in an amount greater than or equal to 15 mg/mL and less than or equal to 25 mg/mL.

5. An injectable composition as in claim 1, wherein the injectable composition is capable of encapsulating the hydrophobic immune-adjuvant at a concentration of greater than or equal to 5 mg per 1 mL of injectable composition.

6. An injectable composition as in claim 1, wherein the molecular weight of each PLGA is greater than or equal to 1400 and less than or equal to 1600.

7. An injectable composition as in claim 1, wherein the molecular weight of the PEG is greater than or equal to 1400 and less than or equal to 1600.

8. An injectable composition as in claim 1, wherein the immunoadjuvant is a small TLR agonists.

9. An injectable composition as in claim 1, wherein the small TLR agonist is selected from the group consisting of imiquimod, resiquimod, 852-A, vesitolimod, azd8848, motolimod, and selgantolimod.

10. An injectable composition as in claim 1, wherein the immunoadjuvant is selected from the group comprising imiquimod, resiquimod, 852-A, vesitolimod, azd8848, motolimod, 2,3 cGAMP, NKTR-262, RG-7854, DSP-0509, BDB-001, LHC-165, BDC-1001, SHR-2150, JNJ-4964, RO-7119929, DN-1508052, VTX-1463, BNT-411, APR-003, PF-4878691, GSK-2245035, RG-7795 (ANA 773, RO 6864018), Epitirimod (R-851), DSP-3025 (AZD-8848), Sotirimod (R-850, S-30594), Telratolimod (3M-052, MEDI-9197), Isatoribine (ANA-245), Loxoribine, ANA-971, ANA-975, RG-7863 (RO6870868), and selgantolimod.

11. An injectable composition as in claim 1, wherein the injectable composition encapsulates the hydrophobic immunoadjuvant at a concentration of greater than or equal to 1 mg per 1 mL of injectable composition.

12. An injectable composition as in claim 1, wherein the injectable composition encapsulates the hydrophobic immunoadjuvant at a concentration of greater than or equal to 1 mg per 1 mL of injectable composition.

13. An injectable composition as in claim 1, wherein the micellar structure comprising poly(lactide-co-glycolide-b-ethylene glycol-b-lactide-co-glycolide) (PLGA-PEG-PLGA) having a lactic acid (LA) to glycolic acid (GA) ratio of less than or equal to 5:1.

14. An injectable composition as in claim 2, wherein the micellar structure comprising poly(lactide-co-glycolide-b-ethylene glycol-b-lactide-co-glycolide) (PLGA-PEG-PLGA) having a lactic acid (LA) to glycolic acid (GA) ratio of less than or equal to 2:1.

15. A method of treating a tumor, comprising:
injecting a composition intratumorally, wherein the composition is a liquid at room temperature and is a gel at 37° C., wherein the composition comprises:
a micellar structure comprising poly(lactide-co-glycolide-b-ethylene glycol-b-lactide-co-glycolide) (PLGA-PEG-PLGA) having a lactic acid (LA) to glycolic acid (GA) ratio of greater than or equal to 3:1; and
an immuno-adjuvant encapsulated within the micellar structure and present within the injectable composition at a concentration of greater than or equal to 1 mg/mL,
determining the position of the composition during injection using CT,
wherein the immune-adjuvant exhibits an extended release profile from the injectable composition over greater than or equal to 24 hours under physiological conditions.

16. The method as in claim 15, further comprising performing a cryoablation concurrently to the injecting.

17. The method as in claim 15, further comprising gelling the composition at a location of the injecting.

18. The method as in any one of 15, further comprising retaining the composition at a location of the injecting.

19. The method as in any one of 15, wherein the injecting occurs at a location internal to a subject.

20. The method as in any one of 15, further comprising degrading the composition at a location internal to a subject.
